(19) United States
(12) Patent Application Publication
(10) Pub. No.: US 2025/0145988 A1
(43) Pub. Date: May 8, 2025

(54) METHODS OF ENRICHING NUCLEIC ACIDS

(71) Applicants: WAGENINGEN UNIVERSITEIT, Wageningen (NL); MSCLS B.V., Den Haag (NL)

(72) Inventors: John Van Der Oost, Renkun (NL); Isabelle Anna Zink, Wageningen (NL); Daniël Christianus Swarts, Bennekom (NL); Max Jan Van Min, Den Haag (NL)

(21) Appl. No.: 18/835,508

(22) PCT Filed: Feb. 1, 2023
(86) PCT No.: PCT/EP2023/052479

§ 371 Date: Aug. 2, 2024

(30) Foreign Application Priority Data
Feb. 2, 2022 (GB) 2201341.1

(51) Int. Cl. C12N 15/10

(52) U.S. Cl. CPC C12N 15/1093

(57) ABSTRACT

Nucleotide sequences of interest, which may be as yet unknown sequences, comprised in a biological sample may often be present in diminishingly small amounts, meaning there are difficulties in detecting, sequencing and identifying these sequences. A method of enrichment of the sequences of interest prior to sequencing of a sample overcomes the problem. In such a method a library of nucleic acid guides is generated from a portion of the sample itself, and the guides are used with a guide-dependent endonuclease such as an Argonaute, usually a prokaryotic Argonaute (pAgo) in a reaction which cleaves the nucleic acids recognised by the guides in another portion of the same sample, but which spares the low abundance sequences for which no guides have been generated. In this way a sample enriched for rarer or low abundance sequences is provided and used in subsequent steps of detection of sequences present, including sequencing of the enriched sample. The method has wide range of application in scientific research of all kinds and forensics where is it necessary to detect the presence of nucleotide sequences and where needed to allow for the detection and/or sequencing of these.

### FIELD OF THE INVENTION

The invention relates to methods of identifying biomarkers in the form of mutations and/or epigenetic changes in the genetic material of biological samples. More particularly the invention concerns methods for selectively fragmenting and enriching certain nucleic acids of known or unknown sequences and low abundance present in samples of nucleic acids.

### BACKGROUND

The sequencing and detection of rare or low-copy number nucleic acids present in samples of nucleic acids continues to present technical challenges. High-copy number nucleic acids outcompete and drain reagents used in amplification and/or sequencing reactions. The rare or low-copy nucleic acid species often remain undetected, or undetectable with the sensitivities of current sequencing technologies, resulting in incomplete sequence data, which in case of certain clinical or research contexts can mean failure to identify clinically relevant biomarkers, thereby confounding diagnoses and genetic studies.

Tumour genotyping allows for the identification of oncogenic mutations responsible for the initiation and maintenance of cancer and mechanisms of resistance to targeted therapeutics. “Noninvasive detection of response and resistance in EGFR-mutant lung cancer using quantitative next-generation genotyping of cell-free plasma DNA “Oxnard, Geoffrey R., et al. (2014)¹ is an example of how non-invasive methods of cancer allele detection can be used to select an effective therapy. Such targeted therapeutics improve outcomes and reduce adverse effects and cost, especially when effective treatment options are identified early in the progression of an aggressive cancer as patient survival rates can diminish quickly over time. Biomarkers obtained from a patient can be used to better understand tumour genetics, susceptibility to drugs, and drug-resistance, as well as an early diagnosis. In some situations biomarkers may reveal a successful treatment regimen and as such may avoid the need for further unnecessary therapy. In addition, the sensitive detection of tumour biomarkers can be used to assess the efficacy of a given treatment and enable the early detection of relapse.

Due to poor health and/or inaccessible tumour location, tumour biopsies are not available from certain patients. Also, tumour biopsies may provide only localized samples which are not representative of the full spectrum of cancer-related mutations. Liquid biopsy (LB) is a minimally invasive alternative technique for testing blood or urine from a patient. The LB yields cell-free circulating tumour DNA (cf-ctDNA) or cell-free circulating tumour RNA (cf-ctRNA). LB can be used as a source of fresh tumour-derived material. Assays can then be used to detect genetic biomarkers and thereby information pertaining to cancer genotypes and the abundance, presence or absence of tumour cells in a patient's body. Circulating tumour DNA tests can thus be used to determine the success of a given therapy and detect disease recurrence early. As such, LB-based testing also promises to enable the discrimination of patients that do and do not require further treatment and significantly improve therapy decisions for those patients that do.

Since ctDNA levels are very low in patients with the small tumours of which ctDNA tests are designed to detect the presence, ctDNA tests require very high sensitivity. Current ctDNA tests are based on very deep sequencing (for instance Cancer Personalized Profiling by deep Sequencing (CAPP-Seq)). In addition, multiple methods have been developed to increase sensitivity, such as polymerase chain reaction (PCR) based methods that, depending on oligonucleotide primer design, can suppress wild type DNA amplification with peptide nucleic acid (PNA)-clamping or digital drop PCR (ddPCR) with and without multiplexed preamplification. Both of these techniques can be used to identify mutant alleles. However, the challenge of these and other existing sensitive genotyping assays, is that biomarkers for undiagnosed cancers are rare mutants, and that their detection is often masked by the wildtype allele which is present in greater abundance. In addition, each patient will have his/her unique tumour specific mutations. A number of techniques designed to detect ctDNA's are therefore personalised assays (for instance: https://www.natera.com/oncology/signatera-advanced-cancer-detection/) and require prior knowledge of to be detected tumour specific mutations.

WO2019/178346 A1 University of Pennsylvania & Wageningen Universiteit discloses a method of enriching a target nucleic acid in a sample comprising contacting the sample with a guide nucleic acid having a sufficiently complementary sequence to a non-target nucleic acid to allow hybridization of the guide nucleic acid and the non-target nucleic acid to form a guide/non-target hybrid; contacting the sample with an endonuclease having an affinity for the guide/non-target hybrid; and amplifying the target nucleic acid. The method is applicable to detecting the presence or absence of cell-free circulating tumour nucleic acids (cf-ctNA) in a sample from a patient. This method relies on prior knowledge of the nucleotide sequence of the target nucleic acid. This therefore limits the ability of the method in that it requires the design and synthesis of guide DNA sequences and that it cannot detect rare or low-copy genetic biomarkers pertaining to cancer phenotypes which have not already been established as such.

Song, J., et al (2020)² is a scientific publication subsequent to but related to WO2019/178346 A1. Song et al., (2020)² describes the application of TtAgo to accomplish a 60-fold enrichment of the known cancer biomarker KRASG12D of known sequence, and ˜100-fold increased sensitivity of Peptide Nucleic Acid (PNA) and Xenonucleic Acid (XNA) clamp PCR, enabling detection of a very low-frequency (<0.01%) these mutant alleles (˜1 copy) in blood samples of pancreatic cancer patients.

He et al., (2017)³ describes a method of PfAgo-mediated nucleic acid detection (PAND). This is the application of PfAgo for detecting SNPs in clinical samples in combination with molecular beacons. The assay is constructed whereby if a nucleic acid of known sequence is cleaved by PfAgo, the cleaved sequence can be utilized by PfAgo to bind and cleave a molecular beacon of complementary sequence resulting in measurable fluorescence, leading to a detection of specific targets. In this way, from ctDNA, human papillomavirus (HPV) and single nucleotide polymorphisms (SNPs) in breast cancer alleles (BRCA1 and rs12516) were detectable when amplified from serum samples.

Liu et al., (2021)¹⁵ describes a single-tube PCR-based PfAgo-directed specific target enrichment and detection method (A-Star). In this application, PfAgo in complex with pre-designed guides of known sequence is added to a PCR reaction containing allele-specific PCR primers and a mixture of SNV-carrying alleles and wild type alleles of known sequence as template. During the denaturation step of the PCR reaction, PfAgo-guide complexes detect and cleave the wild type sequences followed by primer-dependent amplification of uncleaved nucleic acids within the later steps of the PCR reaction. In this way, low frequency (0.01%) of mutant alleles of three known cancer biomarkers (KRAS G12D, PIK3CA and EGFR) were enriched by around 5500-fold in non-complex DNA samples containing a mixture of the respective SNV-carrying allele and the corresponding wild type allele. Furthermore, when performing an additional PCR amplification step prior to the PfAgo-containing PCR amplification reaction, the KRAS G12D mutant allele could be enriched to up to 28-fold and up to 5-fold in DNA purified from patients' blood and tissue samples, respectively.

Wang, et al., (2021)¹² describes a PfAgo-based detection of SARS-CoV-2. Guide DNAs of known sequence are used coupled to molecular beacons and fluorescent signal is monitored. The detection system is able to identify specific single point mutations. These methods therefore enable the depletion of known sequences and the detection of known SNPs. However, since each patient will have his/her unique set of tumour specific markers there is still a need for methods for detecting what may be unknown nucleic acids in samples, or rare, low-copy number nucleic acids in samples of nucleic acid in a sample. Not only is there a need to be able to detect such low abundance and unknown sequences in relation to samples or blood or urine containing cf-RNA or cf-DNA, but also in relation to other samples from a variety of sources containing nucleic acids, e.g. culture samples, environmental samples.

### BRIEF SUMMARY OF THE DISCLOSURE

Accordingly, the present invention provides a method for screening for and/or identifying a nucleotide sequences of interest comprised in nucleic acids of a biological sample, comprising contacting at least a portion of the nucleic acids of the sample with a plurality of nucleic acid guides and a guide-dependent endonuclease, wherein the sequences of the guides align substantially without mismatches with the entirety of, or at least a portion of, nucleotide sequences expected to be present in the sample, wherein sample nucleic acid-endonuclease-guide complexes are formed and have endonuclease activity, and whereby expected nucleic acids in the sample are cleaved, and nucleic acid sequences in the sample which are not sufficiently complementary to any guide sequences are not cleaved.

In another aspect, the invention provides a method for screening for and/or identifying a nucleotide sequence of interest comprised in nucleic acids of a biological sample, comprising contacting at least a portion of the nucleic acids of the sample with a library of nucleic acid guides and a guide dependent endonuclease, wherein the library of guides is obtained or derived from at least another portion of the same or a different sample, wherein the sequences of the guides align substantially without mismatches with the entirety of, or at least a portion of, nucleotide sequences expected to be present in the sample, wherein sample nucleic acid-endonuclease-guide complexes are formed and have endonuclease activity, and whereby expected nucleic acids in the sample are cleaved, and nucleic acid sequences in the sample which are not sufficiently complementary to any guide sequences are not cleaved.

In another aspect the invention provides a method for enriching a collection of unspecified nucleotide sequences from a pool of nucleic acids isolated from a biological sample, comprising contacting the majority of the nucleic acids of the sample with a pool of nucleic acid guide-endonuclease complexes, wherein the sequences of the collection of guides align substantially without mismatches with the entirety of, or at least a portion of, nucleotide sequences expected to be present in the sample, and whereby expected nucleic acids in the sample are cleaved, and unspecified nucleic acid sequences in the sample which are not sufficiently complementary to any guide sequences are not cleaved.

More particular features of each of the aforementioned aspects are explained below.

Hitherto, methods of detecting rare sequences in sample, e.g. mutations or contaminants, have required rationally designed guides based on existing and previously discovered information. Advantageously, the present invention allows for the unbiased detection of rare sequences, e.g. mutations, contamination, in a biological sample without any prior or existing knowledge as to what these rare sequences might be. The invention allows for an entirely free and unbiased discovery of any rare sequences in a biological sample that would otherwise not be detected. As a way of achieving this, the invention harnesses the discriminatory power of guided endonucleases, which are assembled in a massively parallel approach using a guide library that represents substantially all of the sequences in the sample being interrogated. The action of the guided endonucleases on the sample cleaves substantially all of the sequences; optionally all of the sequences which are not of interest, thereby effectively revealing any sequences of interest. These sequences will not be cleaved due to their particular sequences which will not be recognised by any of the guides to the extent of causing cleavage by the endonuclease.

The aforementioned method involves selectively fragmenting nucleotide sequences in the biological sample to identify the nucleotide sequences which are of interest. At least a portion of the sample is contacted with the guide sequences and guide sequence-dependent endonucleases. In preferred aspects, the guide sequences originate from the same sample that a portion of which is contacted with the guides and endonuclease, or the guide sequences originate from a different sample from the sample or sample portion which is contacted with the guides and endonuclease. The mixture of sample, guides and endonuclease results in endonuclease-guide-sample nucleic acid complexes which have endonuclease activity such that nucleic acids in the sample comprising sequences with at least sufficient complementarity to the sequences of the guide sequences are cleaved, and nucleic acid sequences in the sample of interest which are not sufficiently complementary to the guide sequences are not cleaved. Therefore the nucleotide sequences of interest in a sample are produced by the action of endonuclease-guide-sample nucleic acid complexes which cleave and thereby degrade into smaller fragments all of the nucleic acids other than those which are of interest. The sequences of interest in a sample are those which are lacking the necessary degree of complementarity to the library of guides and are therefore preserved from cleavage by a lack of recognition or binding by endonuclease-guide complexes. This is due to the presence of one or more mismatches at one or more positions between a sample nucleic acid and any of the guides.

In accordance with the invention, the selective fragmentation of nucleotide sequences in a biological sample is the way in which one category of sequences are of interest, and are selected for, i.e. “preserved” or “protected” in preference to another category or categories of sequence which are not of interest and which are fragmented by endonuclease digestion. Generally, the sequences which are to be selected for are rarer or in lower abundance or lower copy number than the other sequences which are fragmented in accordance with the invention. The fragmentation is carried out in such a matter that there may be a size differential between the selected or preserved sequences and the sequences which are not of interest. In some aspects, the preserved sequences may be readily separated from the fragmented sequences, e.g. by electrophoresis, amplification and/or capture using a specific probe or marker, and this then allows the sequencing of just sequences of interest.

The invention therefore permits the identification, through the method of selective preservation, optional separation and then optional sequencing, of polynucleotide sequences, hitherto unknown, or infrequently found, in the context of a biological sample. In samples where most of the nucleic acids are of sequences which are not of interest and only a small proportion of the nucleic acids are of interest, sometimes a diminishingly small proportion, perhaps only a single copy, the bulk of nucleic acids mask these sequences of interest when using known methods of amplification and sequencing or other methods of identification. The methods of the invention effectively unmasks the sequence or sequences of interest from the bulk of nucleic acids in the sample which are not of interest.

“Unknown” sequence in the context of the present invention means that the nucleotide sequence of a nucleic acid may not be known, in the sense that it is not already available in a publicly accessible database or other public source. Also, nucleic acids of “unknown sequence” in the context of the present invention include nucleic acids which at the start of performing a method of the invention are not known because no sequencing or other sequence identification step have been undertaken. In other words, the method of the invention starts blind as to the identity of, and/or sequence of, any nucleic of interest which the method reveals or enriches for. However, once a subsequent step of sequencing or probing is carried out on such a sequence, then the nucleotide sequence is plainly known and may correspond to a sequence already known from a sample, publication or database elsewhere.

The methods of the invention also provide for multiplexing, i.e. the detection of multiple sequences of interest in one single analysis.

The methods of the invention therefore permit the revealing of individual nucleotide sequences which may be of interest but which being of such rarity in the original sample and would not otherwise be efficiently and/or reliably observable using known methods. Such individual sequences may comprise mutations or variant sequences, as will be described in more detail below.

The selective fragmentation in a method of the invention is driven by a guide sequence dependent endonuclease which is complexed with a guide sequence. These are described in more detail below. Guides may be provided from a portion of the sample itself, and/or some or all of the guides may be provided from an existing library or libraries. Therefore a person of skill in the art will appreciate that guides may be synthetic as well as obtained from naturally occurring material. Guides may consist of either known or unknown sequences. Guides may comprise unknown sequence variants of known reference sequences. For instance, a guide DNA may consist of a sequence of a human gene. The guide DNA sequence may be known but the sequence may also comprise unknown sequence variants. Mixtures of naturally occurring and synthetic nucleotides may be used. This may occur when it is already known which nucleotide sequences of greater abundance or of a particular type need to be fragmented in order to reveal the rarer sequences of interest, which, as already explained, are of unknown sequence. Thus, a “sequence of interest” in the context of this invention is partly defined as being a polynucleotide of unknown sequence. That is to say, the entire nucleotide sequence including each and every contiguous base may be unknown. In certain situations, the sequence of interest may be a mutant allele of a known sequence, wherein the sequence of the normal or wild type allele is known, but the particular nature and sequence of the mutant allele is not. Therefore, the unknown allele may differ from the known allele in as little as a single base where the difference is a point mutation. Similarly the unknown allele may differ from the known allele in multiplicities of bases, depending on the nature of the mutation, as described in more detail elsewhere herein. In some cases both the sequences of the guide DNA's as well as the sequences of interest may be completely unknown or they may comprise unknown mutations in a known reference sequence. Thus also, a “sequence of interest” may be a variant sequence, wherein the variant sequence differs from a native or wild-type sequence by one or more nucleotide bases, whether contiguous or not. A variant sequence may therefore comprise one or more mutations, as herein defined.

In certain aspects of the invention, none of the sequences of the guides are known or need to be known in order to discover and know the sequences of interest.

In other aspects of the invention none of the guides are synthetic, in the sense that they have not been synthesized, but are obtained by other means, which may be entirely without knowledge of their sequences. Such guides may be copied directly from naturally occurring nucleic acids in a biological sample; optionally involving some amplification or filtering. In this sense, the guides are randomly obtained, rather than rationally designed.

The guides may be orchestrated so that the invention can be applied for the selective fragmentation of individual or both strands in a genomic DNA sample.

In the analysis of an originally double stranded DNA sample, guide DNA sequences can be designed for a defined single strand or for both strands.

Guide DNA sequences can be designed for both the same or different exact genomic positions in either strand. Since mutations will occur in both strands, combinations of guide DNA sequences can be designed to most efficiently detect mutations in sequences of interest. Guide-dependent endonucleases can be used to enrich for sequences of interest. For instance, if universal primer sites have been added to DNA fragments prior to guide-dependent endonuclease-based fragmentation, specific primer binding sites can then be ligated to the ends resulting from the fragmentation. A combination of universal and (multiple different) individual sequence specific primers can be used to selectively amplify sequences in those DNA fragments in which selective fragmentation has occurred.

Tagging or labelling of such fragment ends can be used to physically separate fragmented DNA fragments from other nucleic acids. In this way it is possible to provide a step of enriching target nucleic acids as a pre-treatment or as part of a multistep process of enriching and/or sequencing nucleic acids in accordance with the invention.

Therefore, the design of guide DNA sequences and the desired enrichment strategy are aligned.

For instance, capture can be used after the selective fragmentation step to enrich those strands that guide DNA sequences were designed to selectively fragment.

In DNA fragments to which universal primers have been added, both strands can be used for the selective fragmentation of sequences that are sufficiently complementary to used guide DNA sequences and enrichment of sequences that comprise mutations.

If universal primers have been added to DNA fragments a combination of universal and (multiple different) individual sequence specific primers can be used to selectively amplify sequences in those strands in which selective fragmentation has occurred.

The selective fragmentation of nucleotide sequences as described in the first aspect of the method of the invention should mostly lead in practice to a degree of isolation and purification of nucleotide sequences of interest present in a biological sample. In this way, sequences of interest are easily identified and isolated by their larger relative size compared to the smaller sizes of the nucleic acids comprising sequences which are not of interest and which are the result of guided endonuclease activity. Therefore, in an alternative aspect, the invention provides a method of enriching nucleotide sequences of interest, optionally sequences which are unknown, present in a biological sample, comprising contacting at least a portion of the sample with (a) nucleic acid guides and a guided nuclease to form nucleic acid guide-nuclease complexes, or (b) nucleic acid guide-nuclease complexes, wherein the nucleic acid guide-nuclease complexes have endonuclease activity such that nucleic acids in the sample with sequences with at least sufficient complementarity to the sequences of the guide sequences are cleaved, and nucleic acid sequences in the sample which are not sufficiently complementary to the guide sequences are not cleaved.

The term “sufficient complementarity” may include 100% complementarity between the guide sequence and target portions of the nucleotide sequences being cleaved. However, a lesser degree of complementarity may also be sufficient for the endonuclease activity to take place at the target portions. Therefore “sufficient complementarity” may include complementarity in a range selected from 70% to 100%, 71% to 100%, 72% to 100%, 73% to 100%, 74% to 100%, 75% to 100%, 76% to 100%, 77% to 100%, 78% to 100%, 79% to 100%, 80% to 100%, 81% to 100%, 82% to 100%, 83% to 100%, 84% to 100%, 85% to 100%, 86% to 100%, 87% to 100%, 88% to 100%, 89% to 100%, 90% to 100%, 91% to 100%, 92% to 100%, 93% to 100%, 94% to 100%, 95% to 100%, 96% to 100%, 97% to 100%, 98% to 100% or 99% to 100%.

The term “not sufficiently complementary” in terms of percentage complementarity is mutually exclusive of “sufficiently complementary”. Therefore if the threshold for sufficient complementarity is at least 97.5% for example, the threshold for not sufficiently complementary is less than 97.5%. Possible threshold percentages for distinguishing between “sufficiently complementary” and “not sufficiently complementary” may be any selected from 90%, 91%, 92%, 93%, 94%, 95%, 95.1%, 95.2%, 95.3%, 95.4%, 95.6%, 95.7%, 95.8%, 95.9%, 96%, 96.1%, 96.2%, 96.3%, 96.4%, 96.6%, 96.7%, 96.8%, 96.9%, 97%, 97.1%, 97.2%, 97.3%, 97.4%, 97.6%, 97.7%, 97.8%, 97.9%, 98%, 98.1%, 98.2%, 98.3%, 98.4%, 98.6%, 98.7%, 98.8%, 98.9%, 99%, 99.1%, 99.2%, 99.3%, 99.4%, 99.6%, 99.7%, 99.8% or 99.9%.

Where there is a contacting of at least a portion of the sample with a nucleic acid guide and a guided nuclease to form nucleic acid guide-nuclease complexes, this may involve the simultaneous, separate or sequential mixing of guides, nuclease and sample portion, thereby generating the complexes. Alternatively, there may be a contacting of at least a portion of the sample with already formed nucleic acid guide-nuclease complexes.

In any of the methods of the invention, the nucleotide sequences of interest are preferably of unknown sequence; and/or are of low abundance in the sample. Where there is a nucleotide sequence of unknown sequence and/or low abundance present this may be just a single example of that sequence in the genome of an organism, e.g. a single mutant allele.

The method of any aspect of the invention may further comprise a step of enrichment for nucleotide sequences; preferably wherein the enrichment comprises a capture and/or amplification based enrichment.

Prior or after the step of contacting in the methods of the invention, the sample or portion thereof may be enriched for sequences in at least a portion of interest of the genome of an organism. For example, individual chromosomes may be isolated and there are a number of techniques known in the art for doing this. The sample or a portion thereof may be enriched for sequences of interest in the transcriptome of an organism. For example, a transcriptome isolation kit such as the RiboMinus™ kit of Thermofisher may be used. This enriches the whole spectrum of RNA transcripts in a total RNA sample by degrading the large portion of ribosomal RNA molecules.

Methods of the invention may further comprise an amplification reaction to increase the copy number of nucleotide sequences; preferably wherein the sample or portion thereof is subjected to amplification; optionally to increase the copy number of the nucleotide sequences in a portion of interest of the genome or transcriptome of an organism. In situations where the amount of starting nucleic acid material in the sample is low, the amplification may take place as part of the sample preparation process, prior to the step of contacting with the guided endonuclease.

Methods of the invention may further comprise a capture reaction to increase the relative copy number of the nucleotide sequences in a portion of interest of the genome or transcriptome of an organism.

Both amplification and capture based enrichment can be performed in such a manner that sequences of interest, e.g. mutations in the to be amplified/captured sequences are as efficiently enriched as sequences which are not of interest, i.e. the sequence without mutations. Amplifications can be performed with imperfectly annealing primers and will amplify mutations in sequences in between these primers. Capture may be extensively used to detect mutations and is routinely performed in such a manner that sequences comprising mutations with respect to used capture probes are also efficiently enriched.

Amplification can also be performed in an untargeted manner; a wide variety of whole genome amplification protocols are available that enable the amplification of small amounts of input material.

Whole genome amplification of a small amount of input material for guide DNA sequence generation may be used to generate a larger amount of DNA (and therefore as much DNA as is required for guide DNA generation) but that, with the exception of possible errors generated in the amplification step, the resulting guide DNA sequences will still only comprise the (limited) genetic variation present in the original small amount of input material.

Similarly, the whole genome amplification of a small amount of a sample of interest is expected to result in multiple copies of the originally present sequences. This can help increase the reliability and efficiency with which rare sequence variants can be detected.

In another aspect, the invention provides a method of obtaining and/or identifying a nucleotide sequences of interest comprised in nucleic acids of a biological sample, comprising contacting at least a portion of the nucleic acids of the sample with a library of oligonucleotide guides and guide-dependent nucleic acid binding proteins, wherein the guide-dependent nucleic acid binding proteins do not have nuclease activity and comprise a label or tag, wherein the sequences of the guides align substantially without mismatches with the entirety of, or at least a portion of, nucleotide sequences expected to be present in the sample, and wherein sample nucleic acid/nucleic acid binding protein/guide complexes are formed, and then separating the nucleic acids bound to the complexes from the unbound nucleic acids in the sample, the separated unbound nucleic acids providing the nucleotide sequences of interest. The library of guides may be provided according to any of the methods of the other aspects of the invention described herein. In this aspect of the invention, nucleic acid binding protein/guide complexes bind to, but do not cleave respective nucleic acids in the sample which are other than a nucleotide sequence of interest. The nucleic acid binding protein/guide complexes and nucleic acids bound thereto are separated from unbound sample nucleic acids on the basis of the tag or label. More particular aspects of this invention when not relating to the use of a nuclease are as defined herein in connection with the nuclease aspects of the invention. Advantageously, the non-nuclease method of this invention may be combined with a separate nuclease based aspect of the invention. Or alternatively, the non-nuclease aspect of the invention can be used to enrich nucleotide sequences of interest from samples without employing a selective fragmentation step involving nucleases.

In another aspect, the invention includes a method of selectively suppressing enrichment of nucleic acid in a sample by including in a reaction mixture used to enrich nucleic acid, a library of guides and a nucleic acid binding protein which does not have nuclease activity, wherein the guide library is sufficiently complementary to corresponding nucleic acids in the sample and forms nucleic acid/nucleic acid binding protein/guide complexes. This aspect of the invention may be used alone or in combination with any of the other aspects of the invention defined herein. Also, in this aspect of selectively suppressing enrichment of nucleic acid, the library of guides may be provided according to any of the described methods and other aspects of the invention.

When a method of enrichment suppression is being used according to this aspect of the invention, then one of the fragmentation aspects of the invention as herein defined may be used separately, sequentially or simultaneously on the same sample. For example, the enrichment may be an amplification reaction and the reaction mixture is thereby an amplification reaction mixture. In preferred aspect, the nucleic acid binding protein without nuclease activity is an inactive Argonuate protein.

The source of the sample may be selected from an organism, a cell culture or an environmental sample. Where the organism is a mammal, including a human, the biological sample can be any material derived from the mammal or human, such as blood, urine, tissues, organs, saliva, hair, or any other cells or bodily fluids or secretions. Specimens or biopsy samples arising from diagnostic, therapeutic or surgical procedures may provide suitable sample material. Any kind of cell culture may provide a biological sample, whether entirely or in part, in the sense that a portion of the culture is taken as the sample. The cells may be of prokaryotic or eukaryotic origin. Amongst the prokaryotic cell cultures are bacteria (including cyanobacteria) and archaea. Eukaryotic cell cultures may be any of protist, plant, fungi, algae, or animal, e.g. insect, bird, fish mammalian or human. More complex biological samples may be used, such as those taken from the environment, e.g. water samples, ice samples, soil samples, rock samples. Also within the scope of the invention are samples wherein there is viral or other nucleic acid containing material, which may be at a low level undetectable by current methods. This may include forensic samples. In connection with forensic samples, the genetic material being looked for may be known or unknown, but usually in low abundance or copy number.

The nucleic acid guides may be prepared from a sample of nucleic acid from a first source, and wherein the sample of interest or portion thereof contacted with (a) nucleic acid guides and a guided nuclease, or (b) nucleic acid guide-nuclease complexes, is from a sample of nucleic acid from a second source.

Nucleic acid guides may be prepared for a limited number of alleles from the biological sample of interest; preferably the nucleic acid guides have sufficient complementarity to abundant sequences in the biological sample.

The first source may comprise a normal cell from an animal, and wherein the second source may comprise a volume of blood from an animal; preferably wherein the first and second source is the same individual animal. In this aspect, the methods of the invention can be used to detect rare sequences, i.e. mutant alleles, in circulating tumour DNA (ctDNA). Advantageously, methods of the invention can be used to detect as yet unknown mutations which may correlate to a tumour or cancer type, or a stage or degree of resistance to any kind of therapeutic regimen. The animal is may be a mammal; and in preferred aspects the mammal is a human.

The first source may comprise a normal cell collected from any kind of tissue sample from an organism. The second source may be an aberrant or unusual cell from the same tissue. Without prior knowledge of any particular mutation or variant sequence, methods of the invention can be used to identify a potential genetic basis of any difference between normal and unusual cells in a tissue.

Generally, the first source may be any sample taken from a normal cell, tissue or organism. The second source may be any sample taken from a contrasting corresponding variant cell tissue or organism.

Regarding the nucleic acid guides, these may be prepared from an optionally amplified portion of the nucleic acid sample itself, preferably by (i) fragmenting the sample nucleic acids, (ii) taking a portion of the fragmented nucleic acids, (iii) hybridizing the portion of fragmented nucleic acids to a set of reference probes, wherein the reference probes are optionally shorter than the nucleic acid fragments, (iv) digesting unhybridized single stranded nucleic acid to form double stranded nucleic acid fragment:probe hybrids, and (v) dissociating the double stranded hybrids so that the digested probes provide the single stranded guides. Examples of suitable reference probes include 5′-biotin modified probes (IDT) based on the human genome (RefSeq). Alternatively, (multiplex) PCR amplifications may be used to generate guide DNA sequences. Capture and amplification steps can also be combined. For the generation of each set a separate set of probes and/or PCR primers may be used.

Guides may consist not just of a single set, but a multiplicity of sets of nucleic acid guides may be used, wherein separate portions of the sample may be contacted with respective sets of nucleic acid guide-nuclease complexes. Where a multiplicity of different set of guides are used, each set of guides may have a differing sequence coverage for the nucleic acid sequences in the sample. Therefore the sequences of one set of guides may be different from the other sets of guides.

Although a single guided endonuclease digestion may provide sufficient selective fragmentation or enrichment of nucleotide sequences of interest, any resulting non-cleaved nucleic acid sequences may be pooled and the process repeated using the same or a different combination of guides. An iterative process of selective fragmentation or enrichment may be used to enhance the specificity and accuracy of the method of the invention for identifying rare, unknown alleles.

Ideally, in certain aspects of the invention associated with cell cultures or environmental samples where the objective is to find the presence of rare, and/or unknown sequences of interest, the nucleic acid guides may be prepared from a separate portion of the sample taken from the same source as the portion of the sample which is then reacted with the nucleic acid guide-nuclease complexes. Ideally such a portion comprises a subset of sequences present in the entire sample, for instance a limited amount of (optionally amplified) DNA. In this way, the statistical likelihood is that for a given portion of the sample, this will not contain a rare sequence and so no guide will be formed for this rare sequence. Thereby the rare sequence(s) present in any other portion of the sample will not be selectively fragmented.

The separate portion of the sample may be taken from the source at a first point in time, and the portion of sample reacted with nucleic acid guide-nuclease complexes may be taken at a second, later point in time. Therefore the methods of the invention may operate to discern rare or low copy number sequences arising temporally, e.g. in a cell culture where a contaminant organism may arise, as well as a spatially, e.g. as between cells within a tissue sample at a single time of sample.

Where guides are formed from a portion of the sample and another portion of the same sample is subjected to a method of the invention, nucleic acid guides may comprise a calculated number of equivalents of a double stranded genome known to be present in the source. A minimum number may comprise 1, 2, 3, 4, 5, 6, 7, 8, 9, 10, 15, 20, 25, 30, 35, 40, 45, 50, 55, 60, 65, 70, 75, 80, 85, 90, 100, 200, 250, 500, 750 or 1000 equivalents of a double stranded genome known to be present in the source. A maximum number may comprise 2000, 2500, 3000, 3500, 4000, 4500, 4600, 4700, 4710, 4720, 4730, 4740, 4750, 4760, 4770 or 4780 equivalents of a double stranded genome known to be present in the source. Any of the aforementioned minimum equivalents may be combined with any of the aforementioned maximum equivalents to provide a range of equivalents. For example, when the sample is from a human, the guides may comprise between 1 and about 4800 equivalents of the double stranded human genome.

In other aspects of methods of the invention and wherein guides are prepared from a portion of the sample, the portion of the sample used to prepare such nucleic acid guide fragments may consist of not more than a fraction of the weight of DNA in the sample. Included therefore are portions of the sample which may consist of not more than 0.01%, 0.1%, 1%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the weight of DNA in the sample.

In further aspects of the methods of the invention, wherein guides are prepared from a portion of the sample, nucleotide sequences of the nucleic acid guides may consist of not more than a fraction of the nucleotide sequences present in the sample. Included therefore nucleic acid guides that may consist of not more than 0.01%, 0.1%, 1%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotide sequences present in the sample.

In methods of the invention there is no particular ratio of guide nucleic acid to sample nucleic acid necessary. So, for example, the amount of guide nucleic acids used, as measured by weight, may be less than, the same as, or more than, the amount of nucleic acids in the sample, again as measured by weight.

As already noted, guides are preferably sample derived but some proportion of guides used may be known and/or synthesized. In general however, the invention employs guides which are sample derived because these provide a massively parallel approach to the cleavage of all expected sequences in the sample by the action of the guided endonuclease. There is therefore no need for the sequences of these guides to be known, although they might be “known” in the sense of being from the cells of an organism whose genetic sequences is part of the public knowledge, and if a sample of the nucleic acids from the cells was sequenced then this fact could be confirmed.

In circumstances where a pre-prepared set of guides is used, for example a synthesized set of guides, or where a set of guides prepared from one sample is used on a different sample, then there is the possibility that some of the guide sequences may not find sufficiently complementary sequences in the sample. However, this would not usually be a problem because sequence analysis and/or or a further round of Ago digestion with another set of guides can be used to focus in on the desired nucleic acid sequences in the sample.

Consequently, where in a sample there are rare or low copy sequences, contaminating sequences, or mutations in certain nucleic acids, these sequences are those “of interest” in the context of the present invention. These sequences are therefore “unknown” in terms of their presence or absence in a given sample. The methods of the invention reveal their presence or absence in a sample. When present, these sequences are the sequences of interest and can be sequenced. Therefore a sequence of interest in accordance with the present invention is a sequence which is not necessarily known prior to performing the method of the invention. A sequence of interest in accordance with the present invention may after sequencing be found already to exist in a public database.

However, an important aspect of the present invention is that it offers the means of screening and means of discovering novel sequences within samples of nucleic acid wherein at least a portion of the nucleic acid sequences may be already known, or may become known from carrying out routine whole genome sequencing on a separate portion of the sample from which methods of the invention are applied to. The screening and discovery aspects of the invention are achievable because when guide populations are created blind as to sequence and en masse from a portion of the sample being interrogated, in practice this may results in a small number of guides which comprise a mismatch to some extent with a corresponding nucleic acid sequence in the sample. Such mismatching guides will cause the guide-endonuclease complex at the relevant recognition locus to fail to cleave the nucleic acid at that locus, resulting in an uncleaved and therefore larger nucleic acid fragment than those of the rest of the sample which will mostly be cleaved due to substantially matching guides being present. These larger uncleaved fragments represent the sequences of interest the methods of the invention seek to discover and/or identify.

As well as guides of unknown sequence, some proportion of guides may be used which are of known sequences. This allows for expected sequences which are not of interest to most reliably be cleaved. Therefore, when guides of known sequences are used these can be provided from existing libraries of nucleic acids.

Therefore, whilst some of the sequences of the sample may be known, for example where the sample of interest has already been sequenced, the sequences of rare or low abundance or mutant alleles are not apparent for whatever reason, e.g. from the type or level of sequencing already carried out, then these as yet known sequences are sequences of interest in accordance with the invention.

For assistance with subsequent ligation or sequencing reactions, any guides may be 5′ phosphorylated, using for example T4 polynucleotide kinase.

Based on prior knowledge of unique sequences in a particular region of a genome of an organism, guides may be generated preferentially for this region.

Nucleic acid guides are preferably of a uniform length. Lengths which are of use in the invention may be selected without limitation, from any of the following: 8mers, 9mers, 10mers, 11mers, 12mers, 13mers, 14mers, 15mers, 16mers, 17mers, 18mers, 19mers, 20mers, 22mers, 23mers, 24mers, 25mers, 26mers, 27mers, 28mers, 29mers or 30mers, 31mers, 32mers, 33mers, 34mers, 35mers, 36mers, 37mers, 38mers, 39mers, 40mers, 42mers, 43mers, 44mers, 45mers, 46mers, 47mers, 48mers, 49mers or 50mers.

Nucleic acid guides are preferably DNA, and/or the sample preferably comprises DNA. If the sample comprises RNA then a reverse transcription step can be used as an initial step, together with DNA synthesis to provide a double stranded DNA sample for use in accordance with methods of the invention.

Patient specific sets of guides may be established and used in various ways. Therefore the invention is of utility in connection with some aspects of personalised medicine. For example, periodic monitoring of samples, e.g. blood samples, may allow detection of newly arising biomarkers in ctDNA, thereby providing an early warning test for the possibility of cancer. Where a patient already has a cancer, then periodic monitoring of biomarkers in ctDNA can help monitoring the stage or progression of the cancer. Where a patient is receiving treatment for cancer, then then periodic monitoring of biomarkers in ctDNA can be used as a way of following the progress and efficacy of the treatment. Where a patient has received treatment for cancer, then a periodic monitoring of biomarkers thereafter may be used to confirm remission or spot recurrence.

Also in accordance with the invention, patient specific sets of guides may be generated with amplification or capture based enrichment of defined sequences in an entire genome. Probes used in the generation of patient specific sets may also be used to enrich for defined sequences after the selective fragmentation step. Such probes and/or primers may be used in kits for the generation of patient specific guide DNA sequence in multiple patients. Given the fact that amplification and capture are routinely performed to enrich for sequences comprising (un)known mutations, defined primer/probe sets can be used to generate different patient specific sets in each individual patient. The invention includes kits comprising patient specific sets of guides; and also kits comprising patient specific sets of probes and/or primers. Advantageously, in the sphere of ctDNA tests for diseases animals, particularly humans, patient specific sets of guides (and by extension patient specific sets of probes and/or primers) provides a convenient, cost effective and consistent way of screening out sequences which are not of interest, thereby revealing and allowing identification of the sequences of interest in a sample.

Without wishing to be bound by any particular theory, what appears to follow from the above is that where the sequences of guides in a library are known, then “sequences of interest” in a sample may be those sequences for which there is no corresponding guide or if there is a corresponding guide then there is sufficient mismatch in sequence whereby no cleavage occurs by the relevant guide-endonuclease complex. An advantage of the present invention is that once a library of guides of known sequence is established from a first patient, e.g. for one cancer type or stage, then the same or a modified library can be used on other patient samples in order to determine the present or absence of variant or unusual sequences. The methods of the invention can be used thereby for detection of possible and expected variant sequences of interest, and/or be used for detection of possible yet novel variant sequences of interest. Over time and with numbers of samples of patients being analysed and accumulated, a database can be assembled of possible variant sequences and the sum of knowledge about a particular cancer and its genesis, progression, susceptibility to treatment or resistance to treatment can be increased.

Methods of the invention may be used to identify the presence of genetic biomarkers of unknown sequence in any kind of patient sample, for the indication of any disease that may be associated or correlated with the biomarker. For example:

Identification of biomarkers in patient samples of e.g blood, plasma or urine for any kind of disease condition. There are over 5,000 known genetic conditions, but the molecular basis of these is not known for all of these. There are likely other as yet to be discovered genetic conditions. Methods of the invention may be used to find a known mutation biomarker present in diminishingly small amount in a sample from amongst the 5,000 or so known mutations without needing to use a specific probe. At the same time, new mutations particular for the individual patient may be established and which correlate with a disease state exhibited by the patient. Infection of a patient with a virus or bacterium or parasite can be established from a small volume of sample, even if the infective agent is present in a diminishingly small concentration in the sample, even as little as a single copy of a nucleotide sequence unique to the infective agent and not found in the normal human body.

Where the sample comprises DNA, DNA-targeting nucleases can fragment single stranded DNA and/or (one or both strands of) double stranded DNA. When the sample comprises DNA, the nuclease is preferably an Argonaute, more preferably a prokaryotic Argonaute (pAgo); even more preferably a pAgo from a thermophilic prokaryote.

A range of other possible Argonautes may be used, depending on the nature of the sample. A pAgo selected from Pyrococcus furiosus (PfAgo) or Methanocaldococcus jannaschii (MjAgo) can provide DNA-guided DNA fragmentation. Thermus thermophilus (TtAgo) can provide DNA-guided RNA fragmentation or DNA-guided DNA fragmentation. Aquiflex aeolicus (AaAgo) can provide DNA-guided RNA fragmentation. Thermotoga profunda (TpAgo) can provide RNA-guided DNA fragmentation. Marintoga piexophila (MpAgo) can provide RNA-guided RNA fragmentation or RNA-guided DNA fragmentation (see references in the table below).

There are many Eukaryotic Argonaues (eAgos) and all rely on RNA-guided RNA binding. Some eAgos can cleave RNA as well and so these eAgo can be used to provide RNA-guided RNA fragmentation.

In accordance with methods of the invention, elevated temperatures, i.e. above 50° C.-55° C. are preferred. Therefore methods of the invention may have an upper threshold temperature selected from about 95° C., about 94° C., about 93° C., about 92° C., about 91° C., about 89° C., about 88° C., about 87° C., about 86° C. or about 85° C. This may be combined with a lower threshold temperate of about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. A higher level temperature range of about 70° C. to about 85° C. may be desirable and for such operating temperatures Argonautes from thermophilic bacteria and archaea are preferred.

Cleavage efficiencies and specificity may vary; different nucleases will cleave with different efficiencies and specificities. (Some) expected nucleic acid sequences may therefore remain uncleaved and (some) sequences of interest may be cleaved. As long as sequences of interest are less efficiently cleaved than the expected nucleic acid sequences, the method can be meaningfully applied to enrich for sequences of interest.

So far, no eukaryotic Argonautes (eAgo) have been discovered with an optimum temperature above about 50° C.-55° C. but then eAgo may be used with RNA guides to target RNA rather than DNA.

The invention also provides a method of preparing low abundance nucleotide sequences present in a biological sample, comprising preparing enriched nucleic acids as hereinbefore defined, and then subjecting the enriched nucleic acids to a nucleic acid amplification reaction. Any suitable amplification reaction may be used, such as polymerase chain reaction (PCR), loop mediated isothermal amplification (LAMP), nucleic acid sequence based amplification (NASBA), strand displacement amplification (SDA), self-sustaining sequence replication (3SR) or rolling circle amplification (RCA).

The invention also provides a method of preparing low abundance nucleotide sequences present in a biological sample, comprising preparing enriched nucleic acids as hereinbefore defined, and then subjecting the enriched nucleic acids to a capture step.

The invention also provides a method of sequencing an unknown nucleic acid sequence present in a biological sample, comprising preparing enriched nucleic acids as hereinbefore defined, and then subjecting the enriched nucleic acids to polynucleotide sequencing. Any suitable method of next generation sequencing may be used, whether first, second or third generation sequencing, all of which are well known to a person of skill in the art.

In any of the aforementioned methods of the invention, the unknown nucleic acid sequence may comprise a mutation; for example a mutation selected from one or more of a single nucleotide change, an insertion, a deletion or a duplication compared to a reference sequence; preferably wherein the mutation is a single nucleotide change.

Methods of the invention may be adapted to selectively fragment sequences to reveal rare methylation positions. Prior to contacting the sample nucleic acids with guide sequences and guide sequence dependent endonucleases, either the guide sequences or nucleotide sequences from the biological sample of interest may be treated with a reagent that specifically reacts with methylated or unmethylated base positions so that nucleotide sequences comprising methylated or unmethylated base positions are selectively preserved from guide sequence dependent endonuclease cleavage. A particular approach is bisulfite treatment which converts unmethylated cytosine to uracil: (https://www.activemotif.com/catalog/695/bisulfite-conversion

Examples of methods to enable methylation detection are provided on: https://international.neb.com/tools-and-resources/feature-articles/enzymatic-methyl-seq-the-next-generation-of-methylome-analysis#:˜:text=EM%2Dseq%20is%20the%20only,alternative%20for%20studying%20disease%20states and https://www.epigentek.com/catalog/bisulfite-conversion-and-other-popular-methods-for- measuring-gene-specific-dna-methylation-n-7.html?newsPath=15.

In connection with detection or analysis of epigenetic changes, guide DNA sequences and enrichment strategies are preferably used that cleave and enrich the strand in which methylation is to be detected.

The invention further provides a method as hereinbefore defined, wherein a computer is used in the processing and/or analysis of sequence data.

### BRIEF DESCRIPTION OF THE DRAWINGS

Embodiments of the invention are further described hereinafter with reference to the accompanying drawings, in which:

FIG. 1 is a schematic representation of a patient blood sample containing a mix of circulating healthy DNA and circulating tumour DNA which may contain a single nucleotide variant (SNV).

FIG. 2 is a schematic representation of cells collected from a biopsy sample. Some cells may contain DNA which has an disease-associated SNV in addition to the healthy DNA.

FIG. 3 is a schematic representation of a culture of a microorganism where there is a degree of contamination by another (mixture of) microorganism(s) that might be more abundant than the microorganism to be enriched for.

FIG. 4 is a schematic diagram of one method (via probe capture) of making guide DNAs from the fragmented DNA obtained from a healthy tissue of a patient (not containing the SNV). These guides can then be used for targeting a nucleic acid obtained from a blood sample from the same patient (containing the SNV, not shown).

FIG. 5 is a schematic diagram of other methods of making guide DNAs from the fragmented DNA of a sample.

FIG. 6 is a schematic diagram showing how guide DNAs and pAgos associate to form guide DNA-pAgo complexes.

FIG. 7 is a schematic diagram showing how guide DNA-pAgo complexes work to discriminate between sample DNA containing a mutant allele and sample DNA containing the wild type allele. Adapted from Song et al., 2020².

FIG. 8 is a schematic diagram showing how guide DNA-pAgo complexes are used in separate reactions on the same DNA fragments in order to ensure that any sample fragments with SNVs or mutant alleles are preserved intact.

FIG. 9 is a schematic diagram of the process of enriching a plasmid as described in Experiment 1

FIG. 10 shows the results of Experiment 1 in terms of normalised rolling median coverage of reads per position and percentage of reads assigned to the plasmids.

FIG. 11 is a schematic diagram of the process of enriching a gene from a mixture of two plasmids which differ in that particular gene sequence only, as described in Experiment 2.

FIG. 12 shows the results of Experiment 2 in terms of normalised rolling median coverage of reads per position on the plasmids and percentage of reads assigned to the gene differing between the two plasmids.

### DETAILED DESCRIPTION

Certain features of the disclosed methods of the invention described herein may be in the context of separate embodiments. However, such features may also be provided in any combination in further distinct embodiments.

The disclosure of each patent, patent application, and publication cited or described in this document is incorporated herein by reference, in its entirety.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an” and “the” include the plural.

The term “about” when used in reference to numerical ranges, cut-offs, or specific values is used to indicate that the recited values may vary by up to as much as 10% from the listed value. Some of the numerical values used herein are experimentally determined, and in such circumstances there is inherently a degree of variability. Values stated herein are subject to this inherent variation. Thus, the term “about” may represent variations of ±10% or less, variations of ±5% or less, variations of ±1% or less, variations of ±0.5% or less, or variations of ±0.1% or less from a specified value.

As used herein, the term “mutation” refers to any variation in a nucleic acid sequence compared to a wildtype (wt) nucleic acid sequence, regardless of the frequency of the mutation. The terms “mutation” and “variation” may be used interchangeably. The terms “mutant” and “variant” may also be used interchangeably. Also used herein is the term single nucleotide variant (SNV) which is well known to a person of skill in the art. Also well-known is the term single nucleotide polymorphism (SNP) and this may be used interchangeably with SNV. Included with the term “mutation” are not just single nucleotide base changes, but also insertions, deletions or substitutions, whether contiguous or not, and of any number of polynucleotides. Also included are indels.

A person of skill in the art will understand that the term “low-copy number” or “low-copy” nucleic acid as used herein refers to a species of nucleic acid, for example an allele, a mutant, or a variant of a nucleic acid, that is present in relatively lower proportion than other wild type species of nucleic acid in a population of nucleic acids. That is, the abundance of a low-copy nucleic acid is lower in proportion than the abundance of a non-low-copy nucleic acid in a population of nucleic acids. In one example, a low-copy nucleic acid refers to the fraction or proportion of a mutant allele in a population of nucleic acids containing mutant and non-mutant alleles. A person of skill in the art will further appreciate that enrichment of a low-copy nucleic acid as referred to herein indicates increasing the proportion or the fraction of the low-copy nucleic acid relative to the population of other nucleic acids. The present methods can achieve this result by cleaving and reducing in size just the fragments of abundant nucleic acids in a sample, thereby increasing the relative abundance of the low-copy nucleic acids fragments, and optionally subsequently or simultaneously amplifying the low-copy nucleic acid, thereby further increasing the relative abundance of the low-copy nucleic acid.

In some aspects, the amount of the low abundance nucleic acid is less than about 10% of the total amount of nucleic acid in a sample. In some aspects, the amount of low abundance nucleic acid is less than about 9%, less than about 8%, less than about 7%, less than about 6%, less than about 5%, less than about 4%, less than about 3%, less than about 2%, less than about 1%, or even less than 1% of the amount of the total nucleic acid in the sample. “High abundance” nucleic acids may be defined in terms of the proportion of total nucleic acids in a sample; these proportions being greater than the aforementioned percentages. In the context of the invention “low abundance” nucleic acids do not include any “high abundance nucleic acids” and vice versa.

In the methods disclosed herein cleavage of abundant nucleic acids and amplification of the target nucleic acid can be performed substantially simultaneously. Using thermophilic endonucleases that have cleavage activity at or near a temperature sufficient for isothermal amplification, sequencing, or other detection reactions allows for simultaneously running the cleavage and detection reactions.

In methods of the invention described herein, there is reference to “nucleic acid guides” and “nuclease”. There is also reference to “nucleic acid guide-nuclease complexes”. Generally speaking, the aforementioned terms include the likes of “guide DNA dependent endonucleases”, “guide RNA dependent endonucleases”, “nucleic acid-guided endonucleases”, “nucleic acid guide dependent nucleases”, “nucleic acid-guided enzymes (NAGE)” and “sequence complementarity dependent nucleases”. More particularly, the nucleic acid guides may be comprised of DNA or of RNA. Also, the nucleases or endonucleases are more particularly Argonautes (prokaryotic or eukaryotic), CRISPR-Cas enzymes or other guided nucleases.

In certain methods the possibility exists to use guided nucleases which are inactive in terms of nuclease activity. Such inactive nucleases bind to a target nucleotide sequence but do not cleave it. Tagging or labelling of such inactive nucleases can be used to physically separate bound targets from other non-target nucleic acids. In this way it is possible to provide a step of filtering away non-target nucleic acids as a pre-treatment or as part of a multistep process of enriching nucleic acids in accordance with the invention.

In some aspects of the present disclosure, amplifying a low abundance nucleic acid may employ polymerase chain reaction (PCR), digital drop PCR, loop-mediated isothermal amplification (LAMP), recombinase polymerase amplification (RPA), or any combination thereof. RAMP is a two stage multiplexed amplification process that combines both LAMP and RPA. Amplifying the target nucleic acid can also include, for example, nucleic acid sequence-based amplification (NASBA), self-sustained sequence replication (3 SR), rolling circle (RCA), ligase chain reaction (LCR), strand displacement amplification (SDA), multiple displacement amplification (MDA), or helicase-dependent amplification (HDA).

Whilst isothermal amplification can allow the simultaneous cleavage and amplification of nucleic acids, thermocycling methods can also be used when the amplification process takes place subsequent to nucleic acid cleavage. Amplification of nucleic acids may comprise a polymerase chain reaction (PCR) using primers specific for adapters that have been ligated to the nucleic acids at an earlier stage.

As used herein, the term “in vitro” means that a sample is taken from an organism, tissue or cell and that the method of the invention is carried out on the sample in isolation outside of the organism, tissue or cell from which it has been taken. The term “in vivo” in contrast means that a procedure or method is carried out in a living organism, e.g. human or whole plant. The term “ex vivo” refers to a method or process carried out on tissue from an organism in an environment external to the organism but with minimal alteration of the natural conditions.

The following is a more detailed description of various embodiments of the invention, exemplified by a series of steps.

### Step 1: Sample DNA

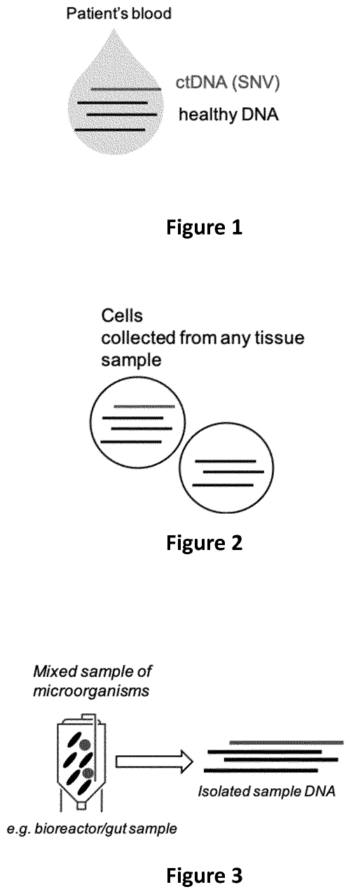

A DNA sample to be analysed in accordance with the invention can be DNA comprised in any sample of interest. Often this sample would originate from a source which is, or comprises, or has comprised, living material. For example, as shown in FIG. 1, the sample can be blood from a patient in which there is circulating normal DNA (healthy DNA) of the patient which is far more abundant than circulating tumour DNA (ctDNA). The ctDNA is expected to contain single nucleotide variations (SNV) of interest and which are not known in terms of genomic location and sequence.

The sample can be of, or from, an organism, e.g. eukaryote or prokaryote. The sample may comprise cells as shown in FIG. 2, and may be collected from a tissue sample. As can be seen in FIG. 2, some of the cells may contain an unknown variant sequence or DNA segment containing an SNV. The sample may be composed of a crude lysate of cells or tissues, or the sample may be a biopsy. Also considered as samples are DNA samples, whether partially or wholly purified. The DNA comprised in any sample may originate from a single organism or a multiplicity of organisms, whether alone or in admixture. FIG. 3 shows a mixed sample of microorganisms wherein there is a smaller proportion of microorganism to be enriched for, the identity or genetic character of which is not necessarily known. DNA comprised in any sample may originate from a single cell or from a multiplicity of cells, whether same or different, or whether the cells are from the same or different tissues or organisms, and thereby the DNA may originate from any mixture of these sources.

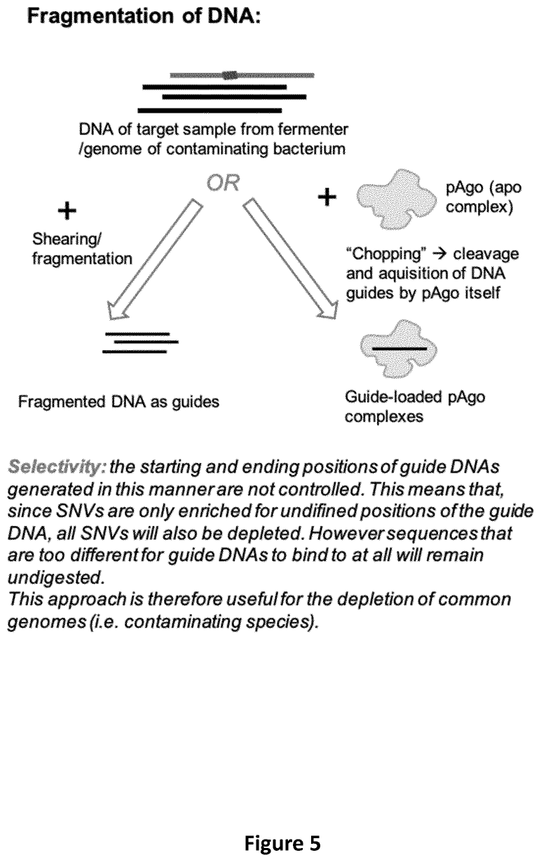

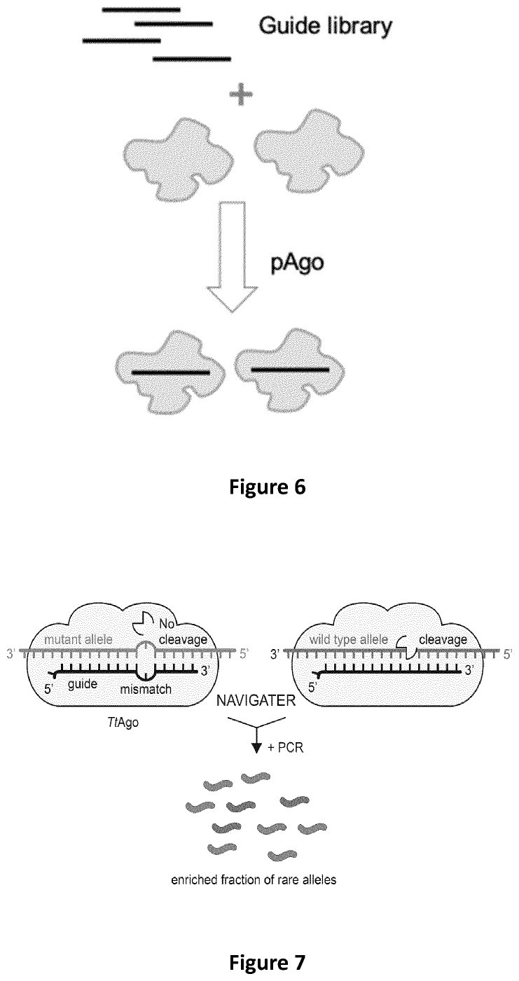

When preparing the sample DNA in accordance with the invention, this may be fragmented as shown schematically in FIG. 5 using any technique, for example enzymatic treatment or mechanical shearing.

In some embodiments, sample DNA fragments may be circularised before subjecting them to pAgo-mediated depletion. Where DNA has been circularised, then an exonuclease treatment can be used after the pAgo-mediated step in order to remove any oligonucleotide DNA sequences that have been linearised as a result of the pAgo-mediated step.

The sample DNA may be subjected to preparation steps, for example the DNA can be amplified before subsequent steps. The sample DNA may originate from reverse transcribed RNA. The sample may consists of RNA.

In accordance with the invention, sequence regions of interest can be examined particularly for the purpose of detecting specific unknown sequences; that is to say known generic sequences or sequence regions can be used to select a pool of nucleic acids within which comprise or are expected to comprise unknown specific sequences. This therefore focuses the method of the invention and helps in a more efficient operation and greater accuracy. For example, particular regions of interest may be enriched from the sample DNA during any step in a method in accordance with the invention. For example, this would be relevant if the interest is in analysis of just coding sequences, wherein the sample DNA might be enriched for the exome before and/or after selective fragmentation.

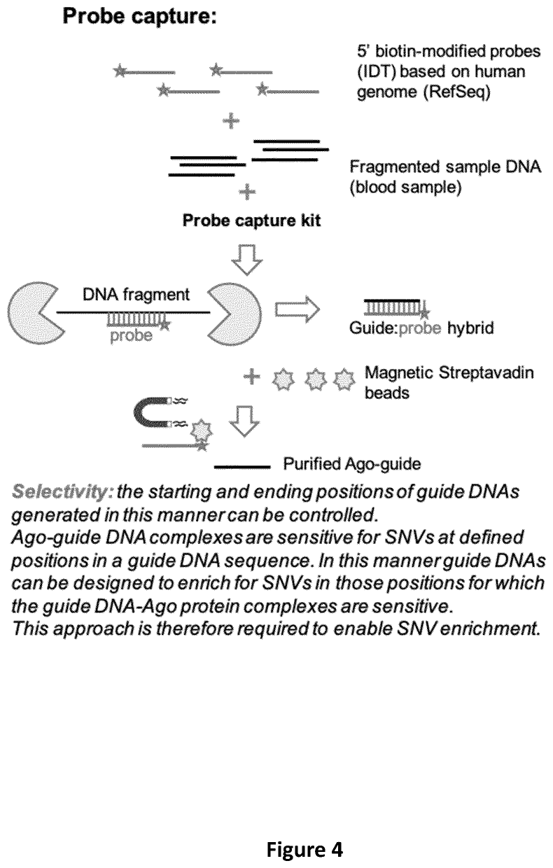

Various sequence or genomic location enrichment strategies can be used to generate pools of DNA oligonucleotide sequences. These may use known starting (5′) and (3′) ending positions. For instance, as shown schematically in FIG. 4, capture probes can be used to capture defined sequences. Single stranded (ss) DNA capture probes can be generated of desired length and sequence. When combined with an exonuclease treatment, such capture probes generate (pools of) DNA sequences with a defined start, end, and uniform length. Various possible exonucleases may be used for treatment of the captured DNA. For example, exonucleases which are available commercially from New England Biolabs (Ipswich, MA, USA). These include exonucleases with dual polarity which means their nuclease activity proceeds both in the 5′ to 3′ and 3′ to 5′ directions. For example Exonuclease V (RecBCD). Where exonuclease activity proceeds in a single direction, 5′ to 3′ exonucleases include for example, T7 Exonuclease, Exonuclease VII (truncated), Lambda Exonuclease and T5 Exonuclease. Where exonuclease activity proceeds in a 3′ to 5′ direction then an example is Exonuclease III (E. coli). Suitable combinations of any of the aforementioned types of exonucleases may be used.

In circumstances where unknown epigenetic changes are desired to be detected in accordance with the invention, the sample DNA may be treated with bisulfite or alternatives thereof. The bisulfite treatment leads to deamination of unmethylated cytosines into uracils, leaving 5-methylcytosines intact which can still then be detected as cytosine, thereby locating the exact positions in a nucleotide sequence which have undergone methylation.

The sample DNA may be prepared to provide a library by end repair and A tailing, adaptor ligation and PCR. This then permits next generation sequencing analysis during any stage during methods of the invention.

Sample DNA, particularly when amplified, can be subdivided into separate subsamples.

### Step 2: Guide DNA Library Generation

Guide DNA may be prepared from any DNA sample of interest that comprises DNA sequences that are complementary to the to-be depleted DNA sequences. For example, where ctDNA is enriched from a blood sample of an animal or a human patient (FIG. 1), then DNA is isolated from a healthy tissue of the same patient and this is then used as the starting material for generating the guide DNA.

Where genomes of a rare microorganism (virus, bacterium, archaeon, yeast, fungus) are enriched from a bioreactor sample of a main (prokaryotic/eukaryotic) cell culture (FIG. 3), the DNA from, for example, a starter culture of the main organism/cell (bacterium, fungus, insect cell, archaeon, yeast, mammalian cell) can be used as a source for the guide DNA. A similar approach can be used for the detection of infecting organisms in a biological sample.

Other opportunities also arise for sourcing samples for generating guide DNA. One is to exploit the already naturally diminishing concentration of rare sequence DNA in a sample which is to be subjected to the method of the invention. In this situation, as shown in the left hand portion of FIG. 5, a limited number of genome equivalents are taken from a portion of the sample, and this limited number of genome equivalents of DNA is used to prepare the guide DNA which is then used on a remaining portion of the sample in accordance with the invention. If needed, this limited number of genome equivalents can be copied. For example, in the situation of enrichment of genomes of a rare bacterium in a bioreactor sample, as shown in the left hand portion of FIG. 5, DNA isolated from the bioreactor sample can serve as guide source sample. Another way of approaching the preparation of guide DNA is to undertake a dilution series of a portion of the DNA sample to be analysed, each dilution in the series then being used to prepare guide DNA. Pools of these guides from different dilutions may be made.

A person of skill in the art will readily appreciate how guide DNA libraries can be generated in a variety of ways. Guide DNA libraries can be generated from living material, biopsies, or isolated DNA. Guide DNA libraries can be amplified prior to their use.

The possibility exists for using commercially generated libraries to prepare guide DNA libraries. For example sets of sequences spanning the sequences of an entire genome could be generated. This would be especially feasible for smaller microbial genomes and these sets could then be used to deplete sequences originating from that genome in mixtures comprising multiple microbial species.

Guide DNA libraries can be fragmented before use by using any known DNA fragmentation strategy. Fragmentation strategies can be used to generate phosphorylated 5′ ends or non-phosphorylated 5′ ends, depending on the nuclease to be used.

DNA used for guide DNA libraries can be amplified with untargeted amplification protocols.

Fragmentation strategies can be used to fragment at defined sequences and therefore to generate known fragment ends.

Guide DNA libraries can be 5′ phosphorylated when required, using T4 polynucleotide kinase (PNK).

As already noted, in order to interrogate samples for methylation changes, guide DNA libraries can be treated with bisulfite, or can be exposed to other chemical/enzymatic treatments in order to specifically convert methylated nucleotides into corresponding nucleotide derivatives.

Guide DNA can be enriched according to any known procedure, such as with DNA capture, PCR or any other enrichment strategy. Enrichment strategies can be used to generate guide DNA nucleotide sequences with known starting and ending positions. Different pools of such guide DNA sequences can be generated, as may be desired.

As shown schematically in FIG. 4, selectivity of a process of guide DNA generation is exemplified. The starting and ending position of guide DNAs are controlled. The Ago-guide DNA complexes are sensitive for SNVs at defined positions in a guide DNA sequence. In this way, guide DNAs can be designed to enrich for SNVs in those positions for which the guide DNA-Ago proteins complexes are sensitive. This approach assists in SNV enrichment.

Where DNA capture is used, it can capture defined sequences. The capture probes which are generated may be of differing length. As already noted, when combined with exonuclease treatment, capture probes can be used to generate guide DNA sequences with defined start, end and length. Different pools of such guide DNA sequences can be generated, as may be desired. An overview of possible exonucleases is available: https://international.neb.com/tools-and-resources/selection-charts/properties-of-exonucleases-and-nonspecific-endonucleases

In contrast, as shown schematically in FIG. 5 in an alternative aspect of the invention, the starting and ending positions of the guide DNAs generated by shearing or fragmentation of a portion of the DNA sample to be analysed, is not controlled. This means that because the SNVs are only enriched for defined positions of the guide DNA, all SNVs will also be depleted. However, sequences that are too different for guide DNAs to bind to at all will remain undigested. This approach is therefore useful for the depletion of common genomes in a sample or culture, so as to enrich rare genomes, e.g. contaminating organisms in the sample or culture.

As shown in FIG. 5, some Apo-pAgo proteins (i.e. pAgo proteins without guides) may elicit off-target cleavage, also known as “chopping”. This assists in the cleavage of larger DNA fragments which are then complexed by the pAgo. The “chopping” characteristic of certain pAgos may help increase the depletion efficiency of the mutated allele (see Swarts et al. (2017)⁷).

In other aspects, ssDNA endonucleases may be used to generate DNA guide sequences. Examples of such ssDNA endonucleases include nuclease P1 or mung bean nuclease to generate guide DNA sequences.

A person of skill in the art will be aware that by using known enrichment/selection strategies, different sets of guide DNA sequences may be generated from a guide DNA sample; optionally an amplified guide DNA sample.

Generally, DNA guides should be short. So, for example, the short 16 nt guides used with TtAgo (see WO2019/178346 A1; also Song et al. (2020)²) tend to form less stable complexes with off-targets (the mutant allele). As described in Song et al. (2020)², the 16 nt guide-TtAgo complexes did not cleave the mutant allele in the depletion step at >75° C., whereas 19 nt guide-TtAgo complexes did. TtAgo guides may be as short as 7 nt or 9 nt (see Wang Y et al (2008)¹³).

### Step 3: Guide DNA-Ago Complex Formation

Guide DNA-Ago proteins can be generated by mixing Argonaute proteins with a guide DNA library or the Argonaute proteins are exposed to a guide DNA library. Schematically this is shown in FIG. 6. Pools of guide DNA-Ago complexes can be made by mixing/exposing Argonautes with respective pools of DNA guides. Mixing can take place in vivo, ex vivo or in vitro, wherein an in vivo mixing may be within a living organism, whether eukaryote or prokaryote. An ex vivo or in vitro mixing may take place within a crude lysate of a cell or tissue or an organism, or in an isolated, possibly partially or wholly purified DNA sample.

Isolated pAgo proteins can be obtained via heterologous expression and then isolated and purified. A usual expression host may be the bacterium Escherichia coli, although any other suitable heterologous host or homologous expression system will be well known to a person of skill in the art. Such isolated pAgo proteins are used in accordance with the methods of the invention as guided endonucleases.

When complexing DNA guides with a pAgo protein, so that the guide-pAgo complex formation reaction can take place effectively, guides should ideally be provided in excess of pAgo. This ensures DNA guide saturation of the pAgos. For example in Song et al. (2020)² a 1:10 ratio of pAgo to DNA guides was used. A 5:1 ratio caused some unspecific cleavage of mutant allele.

DNA guide-pAgo complex formation reaction should ideally be performed at the optimal temperature of the pAgo being used. For example, for TtAgo at a temperature of about 75° C. Likewise, the duration of a guide-pAgo complex formation will depend on the pAgo used. For TtAgo, this is about 20 minutes at 75° C., followed by a 3 minute incubation on ice.

Also known and within the scope of the present invention are pAgos that fragment RNA in a DNA-guide dependent manner, and pAgos that deplete DNA in a RNA-guide dependent manner. Also known and within the scope of the present invention are pAgos which are similar to eukaryotic Argonautes in that they cleave RNA using RNA guides.

### Step 4: Depletion/Enrichment

FIG. 7 (from Song et al., 2020)² is a schematic diagram showing how guide DNA-pAgo complexes are used to cleave and deplete the common sequences (represented by wild type allele) in a sample. pAgos specifically cleave guide-complementary DNA with single nucleotide precision. Some pAgos cleave guide-complementary RNA sequences. Where the guide DNA is entirely complementary to the sample DNA strand then there is endonuclease cleavage by the guide DNA-pAgo complex. In case the guide DNA comprises a mismatch with the sample DNA strand (represented by a mutant allele), there is no or less efficient endonuclease cleavage of that sample DNA strand by the guide DNA-pAgo complex, sparing the rare sequence in the DNA sample and leaving it intact.

The cleavage efficiency of pAgo proteins can depend on the position and type of mismatch between a guide and a target strand. The mismatch can be a single or multiple mismatch. Usefully, there is some variation in mismatch tolerance as between different pAgo proteins and a person of skill in the art will be able to employ these differences constructively in the design of methods and schemes of rare sequence enrichment in accordance with the invention. There are some additional factors which the skilled person can take account of in the design of methods in accordance with the invention. These concern how certain pAgos have different temperature optima and ranges of operation, different mismatch tolerance, and some have differing preference as to guide-length, nature of target sequences and modifications, as well as reaction conditions.

For example, TtAgo is known to be sensitive to mismatches resulting in curtailment of cleavage when the guide DNA has a nucleotide mismatch at position 7-13 (measured from the 5′ end). Furthermore, for TtAgo, the 1^{st} nucleotide in the target sequence should preferably not contain a G, because this enriches the cleavage of mutant alleles, even when there is a mismatch between the guide DNA and target sequence at the aforementioned positions.

For the depletion reaction, pAgo-guide complexes (step 3, see FIG. 6) are used in combination with the sample DNA. When using pre-amplified sample DNA (Step 1, see FIGS. 1-3), the enriched products can be further diluted, for example up to 100-fold or more.

When targets are (partially) single stranded DNA when the Argonaute digestion is performed at elevated temperatures, for example in the range of 60° C. to 95° C. Such temperatures may be greater than 70° C., greater than 80,° C. or greater than 90° C. The actual temperature used depends on thermal stability and activity of selected pAgo. If targets are RNA then lower temperatures may be employed, e.g. in the range 30° C. to 65° C.

A series of separate sequence depletion reactions can be performed, using different sets of DNA guides on respective portions of a subdivided sample of interest.

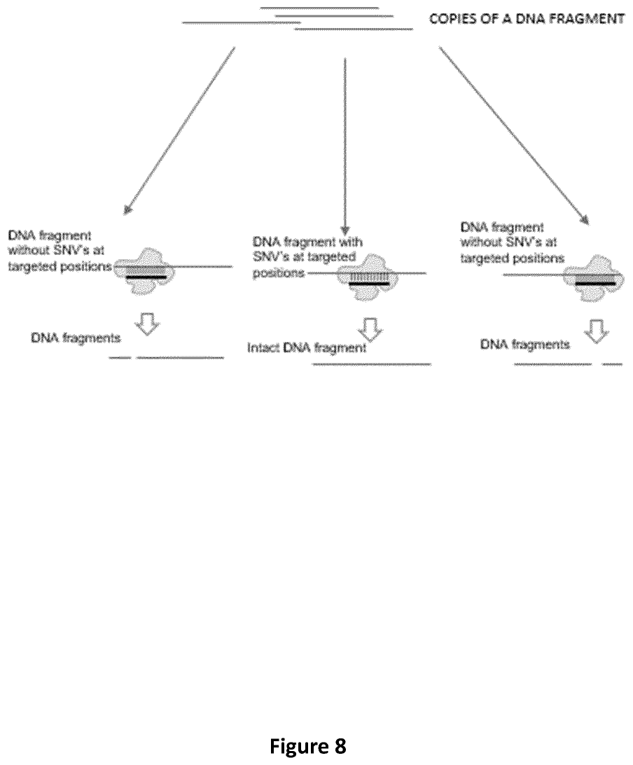

The smaller length of the guide DNA compared to the sample DNA fragments means that a number of individual guide DNAs can map contiguously, substantially end to end, across a given sample DNA fragment, as shown in FIG. 8. Also, individual guide DNAs can map across a given sample DNA fragment, overlapping with each other to varying degrees, and by as much as (n−1) nucleotides, wherein n is the number of nucleotides in the guide DNA. As noted above, pAgos have sensitivity to mismatches across a small number of nucleotides, e.g. 7 nucleotides in the case of TtAgo (guide nucleotides 7-13), and so this creates a window of discrimination. Therefore provided that any overlap between adjacent guide DNA sequences is not less than the window of discrimination, then this opens the possibility of being able to ensure that where there is a sample DNA fragment containing an SNV or mutation, that this sample DNA fragment is not also engaged by another guide DNA fully complementary to a portion of the sample DNA fragment sequence outside of the SNV or mutation. An interrogation scheme using distinct pools of guide DNA sequences in respective reaction vessels can be designed, whereby any sample DNA fragment containing an SNV or mutation will have the possibility of surviving interrogation in one or more of the reaction vessels. In other words, any sample DNA fragment containing an SNV or mutation would be unlikely to be cleaved in all reaction vessels. This aspect of the invention provides much in the way of scope for a person of skill in the art to design suitable pools of guide DNAs to be split amongst chosen numbers of reaction vessels.

So, as shown in FIG. 8, copies of a DNA fragment can be interrogated with Argonaute complexed with multiple independent guide DNA sequences. A pooled digestion of all guide DNA sequences (shown in FIG. 8) would result in the degradation of all DNA fragments. However, performing separate pAgo digestions with different guide DNA sequences in sub-pools, would results in intact DNA fragments that comprise SNVs complementary to any of the targeted positions in a sub-set (one or two) of these sub-pools.

pAgo-guide complexes target ssDNA. pAgo lack dsDNA unwinding activity and so they only target unwound dsDNA. The wildtype depletion reaction (i.e. mutant enrichment reaction) needs to be performed at conditions and temperatures where the sample DNA for interrogation is in single-stranded conformation. For example, when using TtAgo a temperature of about 83 (80-85° C.) is usefully employed.

The duration of pAgo cleavage assays is about an hour, but shorter times of reaction may be used.

The reaction assay can be terminated by adding thermostable proteinase K at 60° C., followed by a 15 minute incubation, or by heat-inactivation of the pAgo complex, for example at about 95° C. for 20 minutes for a TtAgo complex. Optionally, removal of Strep/His-tagged pAgo by affinity chromatography. Another reaction termination may be achieved by the addition of EDTA or another kind of chelating agent, although this may be less desired if the sample is going to be subjected to sequencing. Or, combination of these methods.

A mutant sequence enrichment step can be performed after the pAgo-based sequence depletion by using capture, PCR or any other enrichment approach which will be well known to a person of skill in the art.

If sequencing adaptors have been ligated to DNA sequences prior to the pAgo mediated sample DNA depletion of the invention, then an additional PCR reaction can advantageously be performed to enrich for sample DNA fragments that contain a SNV or mutation and that as a consequence have remained intact.

Many other strategies will be apparent to a person of skill in the art for the purpose of selectively enriching and then sequencing undigested sample DNA fragments.

A number of separate pAgo mediated sample depletions can be pooled in the expectation that the resulting pool of fragments contains one or more sample DNA fragments containing an SNV or mutant allele.

Separate sequence depletion sub-pools can be barcoded prior to pAgo cleavage. NGS sequencing can then be carried out. Sequencing can be performed with any next generation sequencing technology, all of which will be well known to a person of skill in the art. Data analysis can be performed with any appropriate data-analysis tool, and again, these will be well known to a person of skill in the art.

### Enrichment/Sequencing of Uncleaved DNA

The result of a pAgo digestion is single stranded DNA sequences that have either been cleaved and therefore fragmented into smaller sizes, or have remained uncleaved and so remain of original fragment size(s). There are many ways to specifically enrich for and then optionally sequence undigested DNA sequences.

One way is to use primer ligation & PCR. Primers can be added to both ends of the double stranded DNA molecules in the sample prior to pAgo digestion. After pAgo digestion a PCR reaction can be used to amplify just the undegraded DNA nucleotides, which as expected retain both primers.

### Phosphorothioate Nucleotides and Exonucleases

In order to isolate and enrich sample DNA fragments of interest which have survived a pAgo digestion, all of the DNA fragments created by pAgo digestion are themselves digested by nucleases, in particular exonucleases. In order to achieve this, phosphorothioate nucleotide adaptors can be added to both ends of DNA sequences prior to pAgo digestion. The phosphorothioate (PS) bond substitutes a sulphur atom for a non-bridging oxygen in the phosphate backbone of an oligo. This modification renders the internucleotide linkage resistant to nuclease degradation. Phosphorothioate bonds can be introduced between the last 3-5 nucleotides at the 5′-end and/or at the 3′-end of the oligonucleotide to inhibit exonuclease degradation.

In order to be able to inhibit exonucleases, at least five phosphorothioate bonds in a row is recommended. These bonds need to be placed at the end of the DNA fragment corresponding to the polarity of the exonuclease enzyme being used; that is to say at the 5′ end for 5′ to 3′ nucleases, or at the 3′ end for 3′ to 5′ nucleases, and at both ends if the nucleases can initiate at both ends.

Therefore following a pAgo digestion of phophorothiorate modified sample (sub-pool) of DNA fragments, the resulting fragments are subjected to exonucleases. The exonucleases will then digest all DNA fragments which have been generated by pAgo digestion, sparing those which have not been cleaved by pAgo.

### Circularisation & Exonuclease

Sample DNA can first be fragmented and circularised before being subjected to pAgo digestion. Then, the pAgo digestion will linearize the DNA circles comprising DNA sequences complementary to guide DNA sequences. This linearized DNA can in turn be degraded with exonuclease treatment as described above. Unknown sequences of interest which are not subjected to pAgo digestion remain in the form of DNA circles against the background of linear DNA which is then degraded by exonucleases.

### Size Selection

Sample DNA fragments of interest retaining their original length following pAgo digestion can be separated from other DNA fragments of different, i.e. smaller size, by length. A size selection step after pAgo digestion will enrich for undigested DNA fragments. The person of skill in the art will know of many DNA isolation protocols for this purpose. Kits are also commercially available, for example the Monarch® High Molecular Weight DNA Extraction kit (New England Biolabs, Ipswich, MA, USA). In addition, specific electrophoresis equipment is available, for example the Blue Pippin technology (www.sagescience.com/applications/dna-sequencing) that may allow for enrichment of non-cleaved products. The BluePippin systems use pre-cast and disposable agarose gel cassettes. DNA fractions are collected by electro-elution into a buffer-filled well using a branched channel configuration with switching electrodes. The timing of switching is determined by measuring the rate of DNA migration with optical detection of labelled markers.

### Capture Steps

After pAgo degradation, two consecutive capture steps can be used to enrich for intact sample DNA sequences. In a first round, capture probes are used at one end of potential fragmentation sites and the enriched DNA sequences are in turn enriched with capture probes complementary to DNA sequences at the other end of these fragmentation sites. This will result in the capture of intact sequences.

### Ago Proteins

As shown in table 1, prokaryotic Argonaute proteins (pAgos) constitute a diverse group of endonucleases which utilize small nucleic acid guides (DNA or RNA) for sequence-dependent cleavage (or binding) of complementary DNA or RNA targets. (See Hegge et al. (2018)¹⁴). This activity can be repurposed for programmable DNA cleavage (or binding) of desired sequences.

Most characterized pAgos are catalytically active. pAgos can be structurally categorized into “long pAgos” constituted of a N-PAZ-MID-PIWI domains (similar to eukaryotic Argonautes) and “short pAgos” carrying MID-PIWI domains only. 28% of long pAgos have an RNase H-like catalytic centre carrying four conserved amino acids, also known as the catalytic tetrad, which allows them cleave guide bound-target DNA and/or RNA. Short pAgos have a mutated catalytic tetrad and so are catalytically inactive. Short pAgos therefore only bind, but do not cleave a target DNA/RNA. Apart from MjAgo, all other long pAgos characterized to date introduce a single cut between the 10^{th} and 11^{th} nucleotide of the guide-bound single-stranded target, as measured form the 5′-end of the target DNA that is hybridized to the guide. In the case of MjAgo, this has been shown to degrade the target at multiple positions (see ref.⁴).

In the course of pAgo targeting, only the target strand is cleaved: the guide bound to the pAgo remains intact and is therefore reused by pAgo for further target strand binding and cleavage. This allows for multiple turnover of target substrate by individual guide-pAgo complexes. Both active (long) and inactive (short) pAgos differ in their target and guide preferences. A list of biochemically pAgos studied to date is provided in Table 1, including their experimentally verified guide and target requirements and the respective literature.

TABLE 1

| | | | | DNA | RNA | RNA | |
|---|---|---|---|---|---|---|---|
| Organism | Physiology | Domains | In vitro guides | cleavage/binding | cleavage | binding | Literature |
| Methanocaldococcus | hyperthermophilic | N-PAZ-MID- | DNA | dsDNA^{T}, ssDNA, | no | no | [4] |
| jannaschii DSM2661 | | PIWI | | chopping | | | |
| Pyrococcus furiosus | hyperthermophilic | N-PAZ-MID- | DNA | dsDNA^{T}, ssDNA, | no | no | [8] |
| COM1 | | PIWI | | chopping | | | |
| Clostridium butyricum | mesophilic, but | N-PAZ-MID- | DNA, RNA | dsDNA, ssDNA, | no | yes | [5], [6] |
| TK520 | relatively stable | PIWI | | chopping | | | |
| | (cleavage up to | | | | | | |
| | 50° C.) | | | | | | |
| Synechococcus | mesophilic | N-PAZ-MID- | DNA | ssDNA, chopping | no | ? | [18] |
| elongatus PCC7942 | | PIWI | | | | | |
| Clostridium | mesophilic | N-PAZ-MID- | DNA | ssDNA, dsDNA | yes | yes | [19] |
| perfringens | | PIWI | | | | | |
| Intestinibacter | mesophilic | N-PAZ-MID- | DNA | ssDNA, dsDNA | no | ? | [19] |
| bartlettii | | PIWI | | | | | |
| Limnothrix rosea | mesophilic | N-PAZ-MID- | DNA | ssDNA, dsDNA | no | ? | [6] |
| IAMM-220 | | PIWI | | | | | |
| Natronobacterium | mesophilic | OB-N-PAZ- | DNA | dsDNA nicking | yes | yes | [20], [21] |
| gregoryi (inactive?) | | MID-PIWI | | | (preprint | (preprint | |
| | | | | | since | since | |
| | | | | | 2017) | 2017) | |
| Thermus thermophilus | hyperthermophilic | N-PAZ-MID- | DNA, (OH- | dsDNA^{T}, ssDNA, | yes | yes | [17], [22], |
| HB27 and HB8 | | PIWI | RNA^{v}) | chopping | | | [23], [24], |
| Archaeoglobus | hyperthermophilic | MID-PIWI | DNA (RNA) | linear dsDNA | no | yes | [25], [26], |
| fulgidus DSM4304* | | | | binding (as dimer) | | | [27] |
| Kordia jejudonensis* | mesophilic | MID-PIWI | RNA (DNA) | ssDNA/chopping | no | ? | [28] |
| | | | | in combination | | | |
| | | | | with other | | | |
| | | | | nuclease in | | | |
| | | | | operon? | | | |
| Marinitoga piezophila | thermophilic | N-PAZ-MID- | 5′OH-RNA | ssDNA | yes | yes | [29] |
| KA3 | | PIWI | | | | | |
| Thermotoga profunda | thermophilic | N-PAZ-MID- | 5′OH-RNA | SSDNA | ? | ? | [29] |
| | | PIWI | | | | | |
| Rhodobacter | mesophilic | PAZ-MID- | RNA | ssDNA, dsDNA^{T} | no | no | [30], [31] |
| sphaeroides | | PIWI | | | | | |
| ATCC17025/ | | | | | | | |
| ATCC17029 (RNA)* | | | | | | | |
| Aquifex aeolicus | hyperthermophilic | PAZ-MID- | DNA (RNA) | ? | yes | yes | [32] |
| | | PIWI | | | | | |
| Kurthia massiliensis | mesophilic | N-PAZ-MID- | DNA (RNA) | ssDNA, dsDNA, | yes (also | yes | [33], [34] |
| | | PIWI | | chopping | structured | | |
| | | | | | RNA) | | |
| Methanocaldococcus | hyperthermophilic | N-PAZ-MID- | DNA, RNA | ssDNA | no | ? | [35] |
| fervens | | PIWI | | | | | |
| Ferroglobus placidus | hyperthermophilic | N-PAZ-MID- | DNA | ssDNA, dsDNA, | no | ? | [36] |
| | | PIWI | | chopping | | | |
| Clostridium | mesophilic | N-PAZ-MID- | DNA | ssDNA, dsDNA, | no | ? | [37] |
| disporicum | | PIWI | | chopping | | | |
| Maribacter | mesophilic | MID-PIWI | RNA (in | ssDNA binding | no | no | [38] |
| polysiphoniae* | | | complex with | (followed by | | | |
| | | | APAZ-TIR) | NADP hydrolysis | | | |
| | | | | in complex with | | | |
| | | | | APAZ-TIR) | | | |
| Crenotalea | mesophilic | MID-PIWI | RNA (in | ssDNA binding | no | no | [38] |
| thermophila* | | | complex with | (followed by | | | |
| | | | APAZ-TIR) | NADP hydrolysis | | | |
| | | | | in complex with | | | |
| | | | | APAZ-TIR) | | | |
| Sulfolobus islandicus | thermophilic | MID-PIWI | RNA (DNA) | ssDNA binding | no | ? | [39] |
| M.16.4* | | | (in complex | (in complex with | | | |
| | | | with Aga1) | Aga1) followed | | | |
| | | | | by cell membrane | | | |
| | | | | depolymerisation | | | |
| Geobacter | mesophilic | MID-PIWI | RNA (DNA) | ssDNA binding | no | yes | [40] |
| sulfurreducens* | | | (in complex | (followed by | | | |
| | | | with Sir2- | NAD hydrolysis | | | |
| | | | APAZ) | in complex with | | | |
| | | | | APAZ-SIR) | | | |
| Thermus parvatiensis | thermophilic | N-PAZ-MID- | DNA | ssDNA | yes | yes | [41] |
| | | PIWI | | | | | |

? = not tested;
* = inactive PIWI,
ie. no target cleavage, only target binding;
^{T}= high temperature/prior melting of DNA/DNA bubble;
( ) = not preferred;
chopping = guide-independent dsDNA targeting of Apo complex;
^{v}= only shown in vivo

Guided-cleavage or binding of dsDNA in vitro relies on a certain degree of DNA unwinding, and was found to be more efficient in target sequences with low GC content or at elevated temperatures at which dsDNA at least partially occurs in single-stranded conformation (see refs^{5,6,17}). Similarly, guided cleavage or binding of duplexed RNA (i.e. RNA with secondary structure) in vitro relies on a certain degree of unwinding.

Non-specific cleavage of dsDNA by guide-free pAgos, a reaction termed “chopping” is observed for some pAgos in vitro (see Table 1 and refs⁴⁻⁸). The chopping reaction also requires a certain degree of DNA unwinding. The chopping reaction is believed to allow active pAgos to acquire guides autonomously.

Thermostable pAgos have certain advantages when used in the methods of the invention, because the sample DNA can be more readily be denatured by increasing the reaction temperature, thereby reaching a higher level of unwound dsDNA. In case of less stable pAgos, a two-phase system is required, in which initially the target dsDNA is denatured at elevated temperature, after with the temperature is adjusted to the pAgo optimum temperature.

However, for the purposes of the invention, any active pAgo that cleaves a target DNA can be used. Inactive pAgos that identify wild type DNA by binding alone without cleaving could also be used, but this would then require a ‘fishing-out’ of the bound targets.

In more detail, the useful characteristics of pAgos that may be used in various aspects of the invention are as follows:

pAgos for SNV/Rare Sequences Enrichment Through wt/Abundant Target Cleavage

Long-active pAgos can be harnessed for cleavage of guide-matching wild type sequences to enrich for SNV-carrying sequences in a sample. Particular examples of these are:
TtAgo: Probably the best-characterized pAgo, originating from the hyperthermophilic bacterium Thermus thermophilus) (see ref.¹⁷).
   Guided-cleavage: TtAgo uses 5′ P-DNA (or 5′ P-RNA) guides to cleave DNA (see references in Table 1). TtAgo DNA guides can be as short as 7 nt (see ref¹³, but only 16mers have been examined in the context of SNV-enrichment studies so far (see ref.²). 16 bp is also a reasonable length when generating guides, i.e. via fragmentation of probe capture). 16mers would represent a suitable length of guide when using TtAgo for a depletion reaction.
   Chopping; In addition to DNA guided-cleavage of ssDNA by a TtAgo, apo-TtAgo has also been shown to perform guide-independent chopping of dsDNA, resulting in guide acquisition (and cleavage of the complementary ‘passenger’ strand (see ref.⁷)
   Mismatch sensitivity: TtAgo-mediated cleavage was found to be sensitive to mismatches between the guide-target DNA duplex in positions 7-13 (7, 9-13) (see ref.²). Furthermore, for TtAgo the 1^{st} nucleotide in the target sequence should not contain a G, as this enriched cleavage of mutant alleles even in presence of a mismatch at the positions mentioned before. (see ref.²).
PfAgo: PfAgo stems from the hyperthermophilic archaeon Pyrococcus furiosus and can withstand even higher temperatures than TtAgo. These temperatures are up to 100° C., with an optimal temp of 95° C.
   Guided cleavage: Like TtAgo, PfAgo mediates DNA-guided DNA cleavage using 5′-P, 16mer DNA guides (see ref^{7,9,15})
   High mismatch sensitivity (in a 16mer guide); Mismatch sensitivities varies between experimental studies: In ref.³, PfAgo guide complexes were found to be sensitive in positions 3, 4,5, 9-15 (measured from 5′ end of guide) meaning that only positions 1, 2, 6, 7 and 16 are not sensitive to a mismatch, although this varied slightly with different target sequences (ref.³). In ref.¹⁵, efficient discrimination between SNV and wild type alleles was achieved by using guides carrying two consecutive mismatches in positions 10 and 11 or a mismatch at positoin 7 in combination with a mismatch in either position 10 or 11¹⁵. In the method of the present invention, SNV detection might be more stringent when using PfAgo.
   Chopping and cleaved-target-turnover: PfAgo was shown to be capable of chopping dsDNA, thereby acquiring 5′ P-DNA guides (see ref.⁸). As well as chopping, PfAgo was found to be capable of recycling cleavage products into guides, such as short ssDNAs that were generated in the course of DNA-guided PfAgo dsDNA targeting in vitro (see ref.³). In the mentioned study, PfAgo was used in excess of guides (10:1), leaving empty PfAgo complexes in the mix. This indicates that an empty PfAgo which has probably lost its guide or failed to acquire a guide during the “guide DNA-pAgo complex formation step”, can still assemble with previously cleaved wild type DNA used as guides for a second round of cleavage. Hence, this may lead to enhanced cleavage activity of the wild type DNA in the method of the invention, even if guide-pAgo complex formation is inefficient for some guides.
   PfAgo is not sensitive to m6A (dam-)methylation (see ref.⁹). PfAgo was able to cleave m6A-methylated targets where the position of the methylated adenine in the target sequenced matched with the thymine in position 9 of the guide (as measured from the 5′end of the guide). To the best of our knowledge, other methylations (such as m5C) or other guide positions of the m6A-methylation were not tested.
      pAgos for SNV/Rare Sequences Enrichment Via wt/Abundant Target Binding

Short pAgos may be used for binding wild type/abundant DNA, thereby leaving SNV/rare sequences unbound. An advantage can be that those short pAgos are smaller in size, also the guides are smaller. Also, active pAgos could be used (like TtAgo) with shorter guides.
AfAgo: Originates from hyperthermophilic archaeon Archaeoglobus fuldgidus growing in a broad range of temperature of from about 60 to about 95° C. An optimum range of temperature can be from about 60° C. to about 95° C.
   Guided-binding: RNA/DNA guides allow for DNA/RNA binding (see references in Table 1). The lengths of guides may vary. Sequences as short as a seed region (7 nt) can be used as guide for recognition of a similarly long target. However, mostly 12mers-16mer guides may be used. The strongest interaction is with a guide DNA and a RNA target strand.
   Non-guided binding: Apo protein (guide-free) has been shown to bind dsDNA as dimer (see references in Table 1).
   Mismatch sensitivity (measured from 5′ end of guide): RNAg: RNAt (guide/target)=position 4 is sensitive (shows 82% loss of seed-target intereaction-nonwobble A: G), position 3 (wobble, G: U 67% reduction of interaction).

In summary, Argonautes are preferred for use in methods of the invention because there is no PAM requirement with them (which is a feature of DNA-targeting CRISPR-Cas systems). Also Argonautes which employ a short DNA guide are preferred (CRISPR-Cas systems only use RNA guides). With Argonautes, the guides require no flanking sequences (whereas CRISPR-Cas guides have repeat-flanks), hence Argonautes provide for easier acquisition/loading of guides.

In contrast, although CRISPR proteins are less preferred, they may still have utility in methods of the invention. CRISPR-Cas systems are very diverse and can be categorized into Class 1 systems comprising type I, III and IV systems, and Class 2 systems including type II, V and VI systems. All these systems perform RNA-guided targeting. The target nature depends on the type (see Makarova et al., (2020)¹⁰).

CRISPR-Cas Class 1 includes large CRISPR-Cas interference complexes composed of several subunits (up to 13 subunits). In vitro assays using these complexes are cumbersome, as those complexes need to be reconstituted before use.

CRISPR-Cas Class 2 complexes are single-protein systems that can be easily purified and used in in vitro assays (e.g. Type II-Cas9 system, Type V-Cas12a system). Thus, these are CRISPR-Cas proteins which may be used in methods of the invention. Two examples of these are:
Class 2-Type II: 96 nt long RNA guides (SpyCas9), target DNA in a PAM-dependent manner
Class 2-Type V: 55 nt long RNA guides (Cas12a), target DNA in a PAM-dependent manner

The guides of all CRISPR-Cas complexes are of RNA nature and are comprised of a spacer (i.e. target-matching sequence) and a repeat-containing sequence of varying length and at different ends, dependent on the CRISPR type. Hence, whilst the spacer sequence binds the target (like the pAgo guide), the repeat-region is CRISPR-type and array specific and is not variable in that sense. This means that in order to synthesise a guide library, e.g. from RNA or reverse transcribed DNA, according to a method of the invention, the skilled person will need to ligate this (repeat) portion to the guide after/during guide library preparation. Notionally this is comparable to adapter ligation. Also available to a person of skill in the art would be commercially available synthesized guides.

### Other Proteins

Certain other proteins may be used in methods of the invention. The CEL nuclease family of plant DNA endonucleases (CEL1, 2-classical Surveyor nuclease), or the T7 endonuclease I (T7EI) are each used in genome editing and mutation detection workflow.

These nucleases specifically cleave mismatched dsDNA by identifying bulges in the mismatched area. Surveyor nuclease cleaves with high specificity at the 3′ side of any mismatch site in both DNA strands, including all base substitutions and insertion/deletions up to at least 12 nucleotides (see ref.¹¹). Their activity is opposing to the activity of pAgos or CRISPR-Cas which both are sensitive (and therefore do not cleave/bind) to mismatches.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The following experiments were performed using known nucleic acid materials, in order to demonstrate how the method of the invention would operate in practice using a sample of unknown nucleic acid sequence composition. In the experiments, plasmids of known sequence were used for convenience, but equally the experiments could have been performed with additional sequencing steps and sequence analysis in order to achieve the same effect of depletion of certain sequences and enrichment of others.

### Experiment 1: Enrichment of a Plasmid

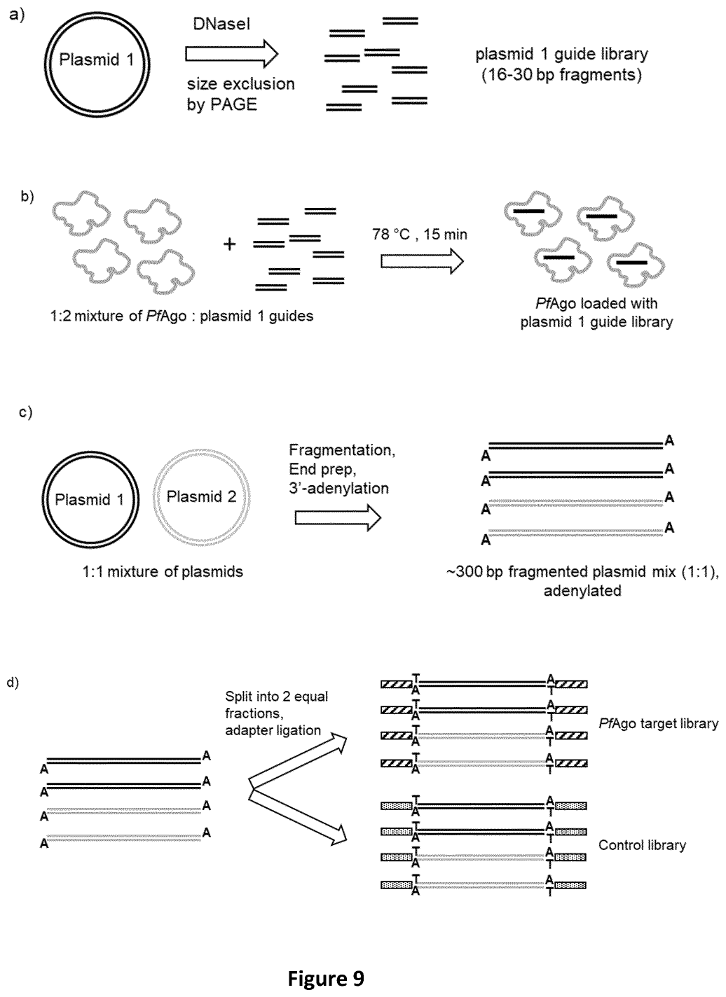

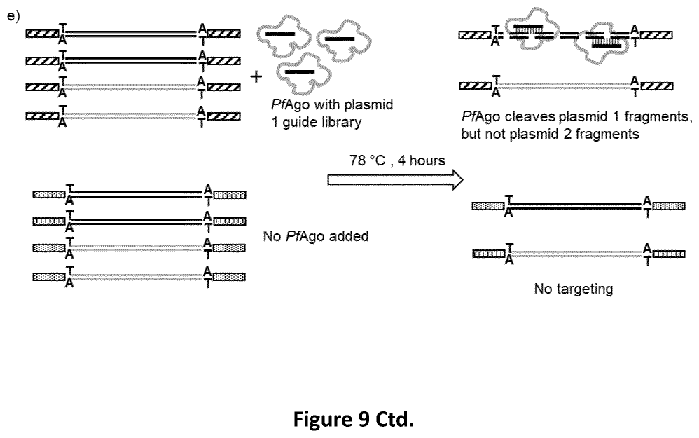

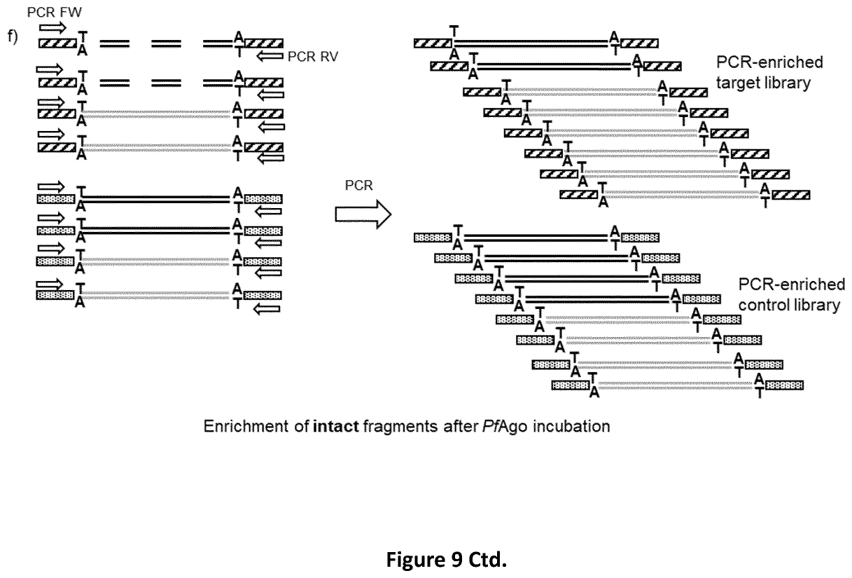

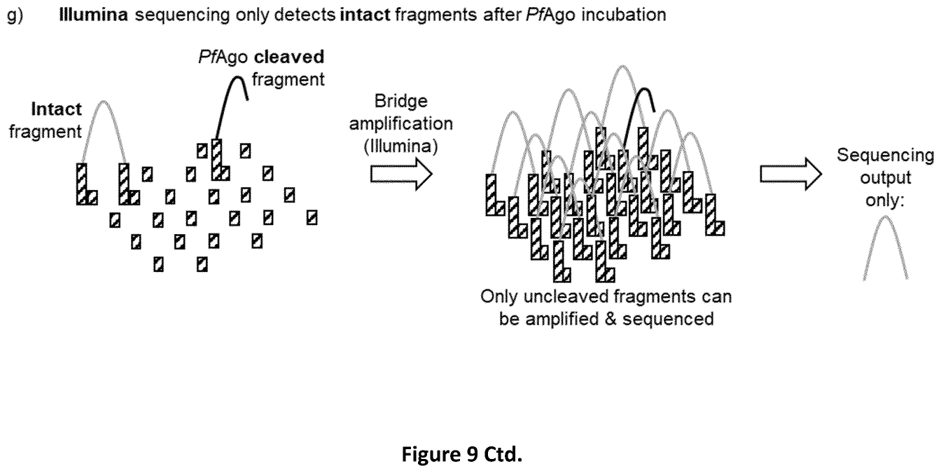

The following is a summary of the experimental steps used in this example:
(i) FIG. 9a shows DNasel being used to generate random fragments from plasmid 1.
(ii) FIG. 9b shows the random DNA fragments (“plasmid 1 guide library”) being loaded onto Pyrococcus furiosus Argonaute (PfAgo).
(iii) FIG. 9c shows how a mixture of plasmid 1 and another genetically different plasmid (plasmid 2) was made. These mixed plasmids were then fragmented and 3′-adenylated.
(iv) FIG. 9d shows the (fragmented and adenylated) plasmid mixture split into two equal fractions and, of each fraction a next generation sequencing library with differently barcoded adapters was generated. This results in a “PfAgo target library” and “control library”.
(v) FIG. 9e shows the PfAgo target library incubated with PfAgo loaded with the “plasmid 1 guide library”. However, no PfAgo was added to the “control library”.
(vi) FIG. 9f shows both target libraries PCR amplified using primers annealing to the Illumina adapters, to enrich next generation sequencing library products that were not cleaved by PfAgo.
(vii) FIG. 9g shows the PCR amplified libraries being sequenced using Illumina sequencing.
(viii) Generated sequences were then mapped to the complete sequences of plasmid 1 & 2.

In more detail, in step (i), plasmid 1 was incubated with 0.033 U DNasel (New England Biolabs, NEB)/μg DNA for 1 minute at 37° C. (FIG. 9a). The reaction was terminated by adding 1.6 U Proteinase K (NEB). The mixture of guides created in this way was separated by 20% Urea-PAGE. DNA fragments of the right size (16-30 bp) were isolated from the PAGE with the ZR small-RNA PAGE Recovery Kit (Zymo Research). These isolated fragments served as “plasmid 1 guide library” to deplete plasmid 1 from a mixture of plasmid 1 and plasmid 2. The DNasel fragments produced in this way are 5′-phosphorylated, being the 5′-modification that is specifically recognized by PfAgo (see reference 8).

In step (ii), the plasmid 1 guide library was then loaded on PfAgo by incubating PfAgo with the guide library in a 1:2 molar ratio in reaction buffer (5 mM MnCl₂, 15 mM Tris-HCl PH 7.6 and 150 mM NaCl), at 78° C. for 15 minutes (see FIG. 9b). The 1:2 ratio of PfAgo:guide is used to achieve PfAgo saturation and thereby suppress its unguided cleavage activity (i.e. by chopping, see for example reference 7).

In step (iii), plasmids 1 and 2 were then mixed in a 1:1 molar ratio (see FIG. 9c). The sizes of plasmid 1 and plasmid 2 are 5026 bp and 4434 bp, respectively. The plasmids are selected so that they are sufficiently different overall in nucleotide sequences, having only one 20 bp sequence and one 16 bp sequence in common. This means that the vast majority of guide sequences will recognize and cleave plasmid 1, but not plasmid 2. The plasmid mixture was fragmented, end prepped, and 3′ ends were adenylated with the xGen™ DNA Library Preparation Kit (IDT) (see FIG. 9c).

In step (iv), the fragments generated in step (iii) were split in two equal fractions and libraries were generated by TA-ligating each library to an adapter set with different barcode sequences (xGen UDI-UMI Adapters; IDT) (see FIG. 9d).

In step (v), library 1 was added to PfAgo loaded with a guide library from plasmid 1 (step (ii)) in a 50:1 molar ratio (PfAgo:target) ratio and incubated for four hours at 78° C. (see FIG. 9e). Library 2 was added to a reaction mix lacking PfAgo and guides but was otherwise treated identical to library 1. The reaction was stopped by adding 1.6 U of Proteinase K (P8107S, NEB), which was subsequently heat inactivated (98° C., 15 min).

In step (vi), to enrich fragments that were not cleaved by PfAgo, the target library was PCR amplified in 25 cycles using primers binding to the ligated adapters (xGen™ Library Amplification Primer Mix (IDT)) with the PCR Master Mix provided with the xGen™ DNA Library Preparation Kit (IDT) according to the protocol from the supplier (see FIG. 9f).

In step (vii), sequencing of PCR-enriched libraries was carried out with the iSeq 100 (Illumina). Illumina sequencing relies on bridge amplification of its library fragments prior to sequencing (see FIG. 9g). This bridge amplification is only possible when both ends of a library fragment have an adapter. Any library fragment cleaved by PfAgo has either one or no adapters at its ends and will therefore not be sequenced. This will result in sequencing of intact fragments only (i.e., not cleaved by PfAgo loaded with the plasmid 1 guide library in step (v).

In step (viii), sequencing reads were quality and adapter trimmed with Trimmomatic v0.39 and then mapped to both plasmids with Bowtie 2 v2.4.1. The number of mapped reads per nucleotide to either of the plasmids was normalized to the total number of reads per library using Samtools v1.6. This results in a percentage of total reads mapped to each plasmid.

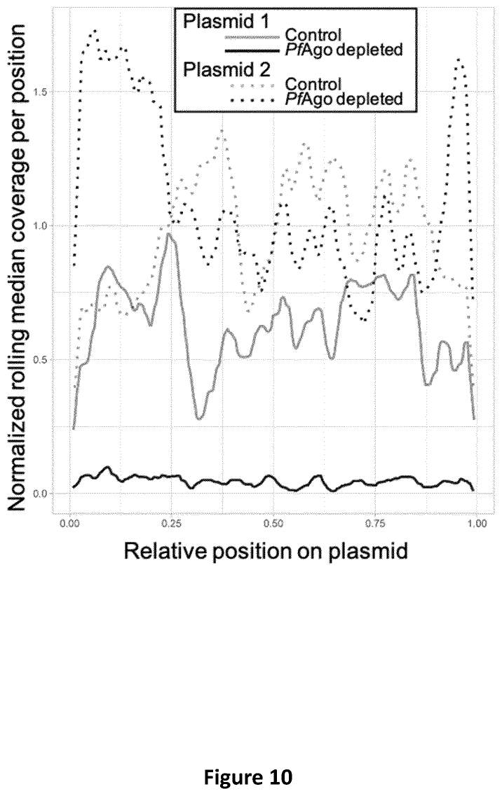

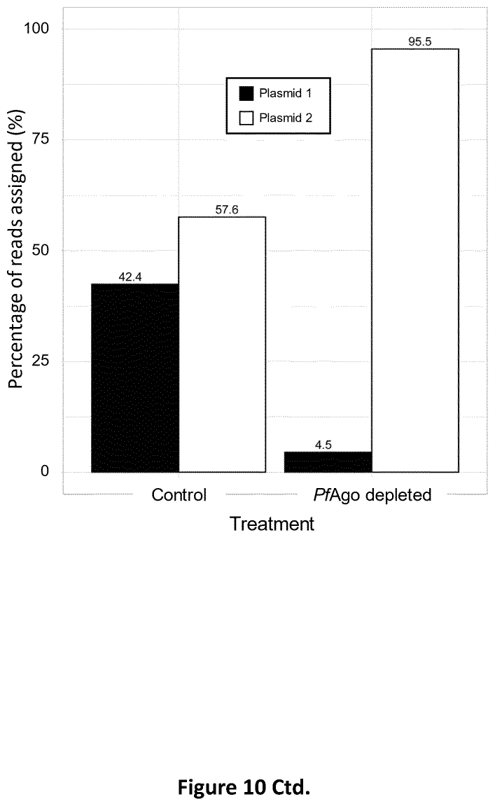

A substantial depletion of plasmid 1 was detected. After PfAgo depletion, the number of reads mapping to plasmid 1 decreased from 42.4% to 4.5%; a 9.4-fold depletion. Whilst the reads mapping to plasmid 2 increased from 57.6% to 95.5%; a 1.7-fold enrichment of plasmid 2 (FIG. 10). This experiment shows how Argonautes when loaded with a randomly generated guide library can be used to deplete the DNA sequence used for guide library generation from pools of DNA in order to enrich untargeted DNA sequences.

It is noted that the sequences of plasmid 1 and plasmid 2 were known in order to most meaningfully analyze the results of the targeted depletion of plasmid 1. However, no sequence information of either plasmid 1 or plasmid 2 was required to design or to perform the described enrichment of plasmid 2 sequences. Plasmid 2 was enriched by just being genetically different from plasmid 1 but no detailed knowledge of either plasmid sequences (and their differences) was required to perform the method.

Generating a PfAgo library with DNA originating from one source was sufficient for its depletion in mixtures in which that DNA occurs together with different DNA from other sources.

It is also noted that in this case the entire plasmid 1 sequence was depleted and the entire plasmid 2 sequence is enriched in generated sequence information.

The increase in sequencing coverage across a region of interest (in this case plasmid 2) depends on the relative abundance of to be depleted sequences.

If, for instance, the to be depleted sequences originally occurred in the same concentration as the sequences of interest, 50% of generated sequencing reads will originate from the sequences of interest without prior PfAgo depletion.

Assuming a 10 fold depletion of to be depleted sequences, the ratio of remaining to be depleted sequences and sequences of interest will have shifted to 0.1 to 1 meaning that 90% of reads will now originate from sequences of interest. The efficiency with which the sequences of interest have been sequenced will thus have increased by 90%/50%=1.8.

If however, the original ratio of to be depleted sequences to sequences of interest is 100 to 1 and a 10 fold depletion is achieved, the % of next generation sequences originating from sequences of interest will have changed from 0.99% to 9.09%. This thus represents a more than 9 fold increase in sequencing efficiency.

The relative concentrations of to be depleted sequences and sequences of interest will depend on the size or respective genomes and their relative abundance.

Especially when looking to analyze smaller (e.g. viral or microbial) genomes in a sample comprising large mammalian or plant genomes, the method of the invention therefore promises to significantly increase the efficiency with which these smaller genomes can be meaningfully sequenced.

### Experiment 2: Enrichment of a Gene

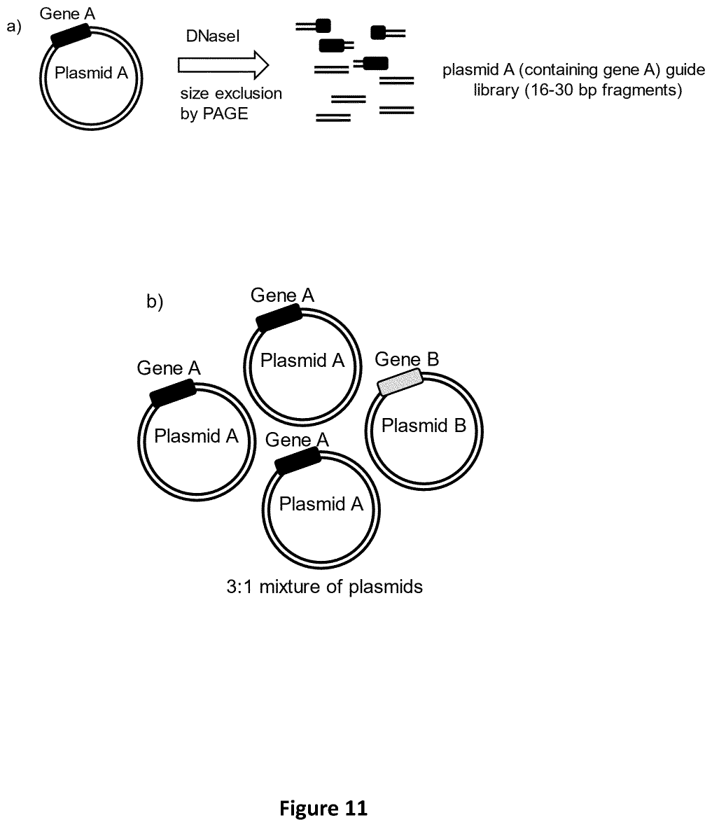

In order to demonstrate that the invention can be used to enrich for a single gene sequence in a mixture of two plasmids that only differ in that gene, a second experiment was performed. This second experiment is a variation on experiment 1 and relevant deviations of the workflow from experiment 1 are schematically depicted in FIG. 11.

Experiment 2 was carried out as described for experiment 1, but with the following adjustments:
(i) Plasmid A was incubated with 0.025 U DNasel (NEB)/μg DNA for 1 minute at 37° C. (see FIG. 11a).
(ii) Two plasmids were used that have identical backbones and either contain a gene A (1395 bp) or a gene B (1398 bp), with no sequence identity, FIG. 11b; these plasmids were mixed in a 3:1 molar ratio (plasmid A:plasmid B) (see FIG. 11b).

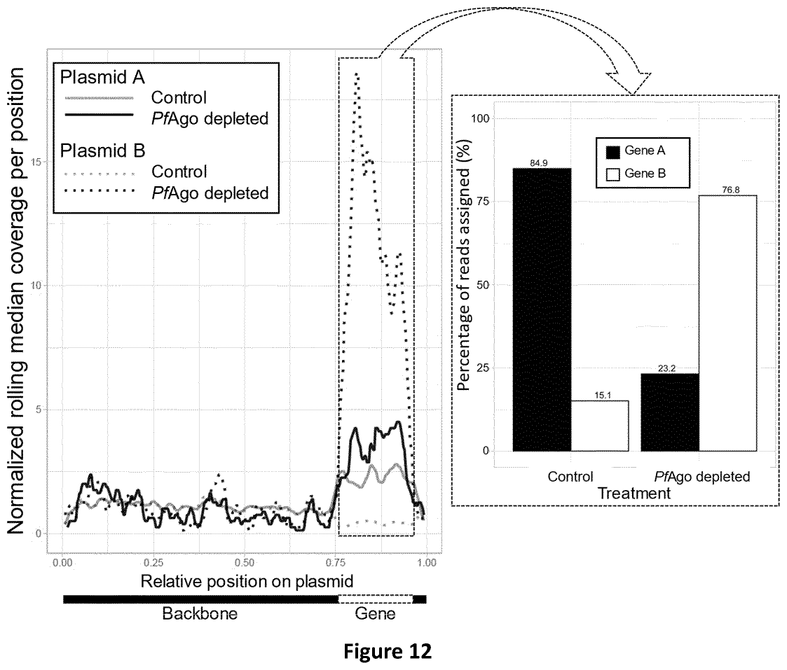

FIG. 12 shows how there was a substantial enrichment of gene B: the fraction of reads mapping to gene B of plasmid B increases from 15.1% to 76.8% after the library was treated with PfAgo loaded with a plasmid A guide library; this is a 5.1-fold enrichment. The reads mapping to gene A were 84.9% prior to PfAgo treatment, this was reduced to 23.2% afterwards; yielding a 3.7-fold depletion. These results indicate that the method is also suitable to enrich a 1398 bp DNA sequence using a guide library generated from a 6758 bp DNA sequence carrying a genetically different sequence in the place of the 1398 bp DNA sequence. This implies that this method can be used to enrich rare sequences in a mixture of otherwise identical DNA sequences.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### REFERENCES

⁽¹⁾ Oxnard G R, Paweletz C P, Kuang Y, Mach S L, O'Connell A, Messineo M M, Luke J J, Butaney M, Kirschmeier P, Jackman D M, Janne P A. Noninvasive detection of response and resistance in EGFR-mutant lung cancer using quantitative next-generation genotyping of cell-free plasma DNA. Clin Cancer Res. 2014 Mar. 15; 20(6):1698-1705. doi: 10.1158/1078-0432.CCR-13-2482.
⁽²⁾ Song J, Hegge J W, Mauk M G, Chen J, Till J E, Bhagwat N, Azink L T, Peng J, Sen M, Mays J, Carpenter E L, van der Oost J, Bau H H. Highly specific enrichment of rare nucleic acid fractions using Thermus thermophilus argonaute with applications in cancer diagnostics. Nucleic Acids Res. 2020 Feb. 28; 48(4):e19. doi: 10.1093/nar/gkz1165.
⁽³⁾ He R, Wang L, Wang F, Li W, Liu Y, Li A, Wang Y, Mao W, Zhai C, Ma L. Pyrococcus furiosus Argonaute-mediated nucleic acid detection. Chem Commun (Camb). 2019 Oct. 31; 55(88):13219-13222. doi: 10.1039/c9cc07339f.
⁽⁴⁾ Zander A, Willkomm S, Ofer S, van Wolferen M, Egert L, Buchmeier S, Stöckl S, Tinnefeld P, Schneider S, Klingl A, Albers S V, Werner F, Grohmann D. Guide-independent DNA cleavage by archaeal Argonaute from Methanocaldococcus jannaschii. Nat Microbiol. 2017 Mar. 20; 2:17034. doi: 10.1038/nmicrobiol.2
⁽⁵⁾ Hegge J W, Swarts D C, Chandradoss S D, Cui T J, Kneppers J, Jinek M, Joo C, van der Oost J. DNA-guided DNA cleavage at moderate temperatures by Clostridium butyricum Argonaute. Nucleic Acids Res. 2019 Jun. 20; 47(11):5809-5821. doi: 10.1093/nar/gkz306.
⁽⁶⁾ Kuzmenko A, Yudin D, Ryazansky S, Kulbachinskiy A, Aravin A A. Programmable DNA cleavage by Ago nucleases from mesophilic bacteria Clostridium butyricum and Limnothrix rosea. Nucleic Acids Res. 2019 Jun. 20; 47(11):5822-5836. doi: 10.1093/nar/gkz379.
⁽⁷⁾ Swarts D C, Szczepaniak M, Sheng G, Chandradoss S D, Zhu Y, Timmers E M, Zhang Y, Zhao H, Lou J, Wang Y, Joo C, van der Oost J. Autonomous Generation and Loading of DNA Guides by Bacterial Argonaute. Mol Cell. 2017 Mar. 16; 65(6):985-998.e6. doi: 10.1016/j.molcel.2017.01.033.
⁽⁸⁾ Swarts D C, Hegge J W, Hinojo I, Shiimori M, Ellis M A, Dumrongkulraksa J, Terns (8) R M, Terns M P, van der Oost J. Argonaute of the archaeon Pyrococcus furiosus is a DNA-guided nuclease that targets cognate DNA. Nucleic Acids Res. 2015 May 26; 43(10):5120-9. doi: 10.1093/nar/gkv415.
⁽⁹⁾ Enghiad B, Zhao H. Programmable DNA-Guided Artificial Restriction Enzymes. ACS Synth Biol. 2017 May 19; 6(5):752-757. doi: 10.1021/acssynbio.6b00324.
⁽¹⁰⁾ Makarova K S, Wolf Y I, Iranzo J, Shmakov S A, Alkhnbashi O S, Brouns S J J, Charpentier E, Cheng D, Haft D H, Horvath P, Moineau S, Mojica F J M, Scott D, Shah S A, Siksnys V, Terns M P, Venclovas Č, White M F, Yakunin A F, Yan W, Zhang F, Garrett R A, Backofen R, van der Oost J, Barrangou R, Koonin E V. Evolutionary classification of CRISPR-Cas systems: a burst of class 2 and derived variants. Nat Rev Microbiol. 2020 February; 18(2):67-83. doi: 10.1038/s41579-019-0299-x.
⁽¹¹⁾ Qiu P, Shandilya H, D'Alessio J M, O'Connor K, Durocher J, Gerard G F. Mutation detection using Surveyor nuclease. Biotechniques. 2004 April; 36(4):702-7. doi: 10.2144/04364PF01
⁽¹²⁾ Wang F, Jun Y, Ruyi H, Xiao Y, Shuliang C, Yang L, Longyu W, Aitao L, Linlin L, Chao Z, Lixin M, PfAgo-based detection of SARS-CoV-2. Biosens Bioelectron 2021 Apr. 1; 177:112932. doi: 10.1016/j.bios.2020.112932. Epub 2020 Dec. 28
⁽¹³⁾ Wang Y, Juranek S, Li Haito, Sheng Gang, Tuschl T & Patel D Structure of an argonaute silencing complex with a seed-containing guide DNA and target RNA duplex. Nature 456, pages 921-926 (2008)
⁽¹⁴⁾ Hegge J W, Swarts D C, van der Oost J. Prokaryotic Argonaute proteins: novel genome-editing tools? Nat Rev Microbiol. 2018 January; 16(1):5-11. doi: 10.1038/nrmicro.2017.73
⁽¹⁵⁾ Liu Q, Guo X, Xun G, Li Z, Chong Y, Yang L, Wang H, Zhang F, Luo S, Cui L, Zhao P, Ye X, Xu H, Lu H, Li X, Deng Z, Li K, Feng Y. Argonaute integrated single-tube PCR system enables supersensitive detection of rare mutations. Nucleic Acids Res. 2021 Jul. 21; 49(13):e75. doi: 10.1093/nar/gkab274.
⁽¹⁶⁾ Collias D, Beisel C L. CRISPR technologies and the search for the PAM-free nuclease. Nat Commun. 2021 Jan. 22; 12(1):555. doi: 10.1038/s41467-020-20633-y.
⁽¹⁷⁾ Swarts, D. C., Jore, M. M., Westra, E. R., Zhu, Y., Janssen, J. H., Snijders, A. P., Wang, Y., Patel, D. J., Berenguer, J., Brouns, S., & van der Oost, J. (2014). DNA-guided DNA interference by a prokaryotic Argonaute. Nature, 507(7491), 258-261. https://doi.org/10.1038/nature12971
⁽¹⁸⁾ Olina A, Kuzmenko A, Ninova M, Aravin A A, Kulbachinskiy A, Esyunina D. Genome-wide DNA sampling by Ago nuclease from the cyanobacterium Synechococcus elongatus. RNA Biol. 2020 May; 17(5):677-688. doi: 10.1080/15476286.2020.1724716
⁽¹⁹⁾ Cao Y, Sun W, Wang J, Sheng G, Xiang G, Zhang T, Shi W, Li C, Wang Y, Zhao F, Wang H. Argonaute proteins from human gastrointestinal bacteria catalyze DNA-guided cleavage of single- and double-stranded DNA at 37° C. Cell Discov. 2019 Jul. 30; 5:38. doi: 10.1038/s41421-019-0105-y
⁽²⁰⁾ Lee K Z, Mechikoff M A, Kikla A, Liu A, Pandolfi P, Fitzgerald K, Gimble F S, Solomon K V. NgAgo possesses guided DNA nicking activity. Nucleic Acids Res. 2021 Sep. 27; 49(17):9926-9937. doi: 10.1093/nar/gkab757
⁽²¹⁾ DNA-dependent RNA cleavage by the Natronobacterium gregoryi Argonaute Sunghyeok Ye, Taegeun Bae, Kyoungmi Kim, Omer Habib, Seung Hwan Lee, Yoon Young Kim, Kang-In Lee, Seokjoong Kim, Jin-Soo Kim, BioRxiv, 2017, doi: https://doi.org/10.1101/101923
⁽²²⁾ Wang Y, Juranek S, Li H, Sheng G, Wardle G S, Tuschl T, Patel D J. Nucleation, propagation and cleavage of target RNAs in Ago silencing complexes. Nature. 2009 Oct. 8; 461(7265):754-61. doi: 10.1038/nature08434.
⁽²³⁾ Parker J S, Roe S M, Barford D. Crystal structure of a PIWI protein suggests mechanisms for siRNA recognition and slicer activity. EMBO J. 2004 Dec. 8; 23(24):4727-37. doi: 10.1038/sj.emboj.7600488.
⁽²⁴⁾ Parker J S, Parizotto E A, Wang M, Roe S M, Barford D. Enhancement of the seed-target recognition step in RNA silencing by a PIWI/MID domain protein. Mol Cell. 2009 Jan. 30; 33(2):204-14. doi: 10.1016/j.molcel.2008.12.012.
⁽²⁵⁾ Parker J S, Roe S M, Barford D. Structural insights into mRNA recognition from a PIWI domain-siRNA guide complex. Nature. 2005 Mar. 31; 434(7033):663-6. doi: 10.1038/nature03462.
⁽²⁶⁾ Ma J B, Yuan Y R, Meister G, Pei Y, Tuschl T, Patel D J. Structural basis for 5′-end-specific recognition of guide RNA by the A. fulgidus Piwi protein. Nature. 2005 Mar. 31; 434(7033):666-70. doi: 10.1038/nature03514
⁽²⁷⁾ Golovinas E, Rutkauskas D, Manakova E, Jankunec M, Silanskas A, Sasnauskas G, Zaremba M. Prokaryotic Argonaute from Archaeoglobus fulgidus interacts with DNA as a homodimer. Sci Rep. 2021 Feb. 25; 11(1):4518. doi: 10.1038/s41598-021-83889-4
⁽²⁸⁾ Kim S Y, Jung Y, Lim D. Argonaute system of Kordia jejudonensis is a heterodimeric nucleic acid-guided nuclease. Biochem Biophys Res Commun. 2020 May 7; 525(3):755-758. doi: 10.1016/j
⁽²⁹⁾ Kaya E, Doxzen K W, Knoll K R, Wilson R C, Strutt S C, Kranzusch P J, Doudna J A. A bacterial Argonaute with noncanonical guide RNA specificity. Proc Natl Acad Sci USA. 2016 Apr. 12; 113(15):4057-62. doi: 10.1073/pnas
⁽³⁰⁾ Lisitskaya L, Petushkov I, Esyunina D, Aravin A, Kulbachinskiy A. Recognition of double-stranded DNA by the Rhodobacter sphaeroides Argonaute protein. Biochem Biophys Res Commun. 2020 Dec. 17; 533(4):1484-1489. doi: 10.1016/j.bbrc.2020
⁽³¹⁾ Olovnikov I, Chan K, Sachidanandam R, Newman D K, Aravin A A. Bacterial argonaute samples the transcriptome to identify foreign DNA. Mol Cell. 2013 Sep. 12; 51(5):594-605. doi: 10.1016/j.molcel.2013.08.014
⁽³²⁾ Yuan Y R, Pei Y, Ma J B, Kuryavyi V, Zhadina M, Meister G, Chen H Y, Dauter Z, Tuschl T, Patel D J. Crystal structure of A. aeolicus argonaute, a site-specific DNA-guided endoribonuclease, provides insights into RISC-mediated mRNA cleavage. Mol Cell. 2005 Aug. 5; 19(3):405-19. doi: 10.1016/j.molcel.2005
⁽³³⁾ Liu Y, Li W, Jiang X, Wang Y, Zhang Z, Liu Q, He R, Chen Q, Yang J, Wang L, Wang F, Ma L. A programmable omnipotent Argonaute nuclease from mesophilic bacteria Kurthia massiliensis. Nucleic Acids Res. 2021 Feb. 22; 49(3):1597-1608. doi: 10.1093/nar/gkaa1278
⁽³⁴⁾ Kropocheva E, Kuzmenko A, Aravin A A, Esyunina D, Kulbachinskiy A. A programmable pAgo nuclease with universal guide and target specificity from the mesophilic bacterium Kurthia massiliensis. Nucleic Acids Res. 2021 Apr. 19; 49(7):4054-4065. doi: 10.1093/nar/gkab182
⁽³⁵⁾ Chong, Y., Liu, Q., Huang, F. et al. Characterization of a recombinant thermotolerant argonaute protein as an endonuclease by broad guide utilization. Bioresour. Bioprocess. 2019 Jun. 5; 6, 21. doi.org/10.1186/s40643-019-0254-8
⁽³⁶⁾ Guo X, Sun Y, Chen L, Huang F, Liu Q, Feng Y. A Hyperthermophilic Argonaute From Ferroglobus placidus With Specificity on Guide Binding Pattern. Front Microbiol. 2021 Jun. 9; 12:654345. doi: 10.3389/fmicb.2021.654345.
⁽³⁷⁾ Sun S, Xu D, Zhu L, Hu B, Huang Z. A Programmable, DNA-Exclusively-Guided Argonaute DNase and Its Higher Cleavage Specificity Achieved by 5′-Hydroxylated Guide. Biomolecules. 2022 Sep. 21; 12(10):1340. doi: 10.3390/biom12101340
⁽³⁸⁾ Koopal B, Potocnik A, Mutte S K, Aparicio-Maldonado C, Lindhoud S, Vervoort J J M, Brouns S J J, Swarts D C. Short prokaryotic Argonaute systems trigger cell death upon detection of invading DNA. Cell. 2022 Apr. 28; 185 (9): 1471-1486.e19. doi: 10.1016/j.cell.2022.03.012.
⁽³⁹⁾ Zeng Z, Chen Y, Pinilla-Redondo R, Shah S A, Zhao F, Wang C, Hu Z, Wu C, Zhang C, Whitaker R J, She Q, Han W. A short prokaryotic Argonaute activates membrane effector to confer antiviral defense. Cell Host Microbe. 2022 Jul. 13; 30(7):930-943.e6. doi: 10.1016/j.chom.2022.04.015.
⁽⁴⁰⁾ Zaremba M, Dakineviciene D, Golovinas E, Zagorskaitė E, Stankunas E, Lopatina A, Sorek R, Manakova E, Ruksenaite A, Silanskas A, Asmontas S, Grybauskas A, Tylenyte U, Jurgelaitis E, Grigaitis R, Timinskas K, Venclovas Č, Siksnys V. Short prokaryotic Argonautes provide defence against incoming mobile genetic elements through NAD+ depletion. Nat Microbiol. 2022 November; 7(11):1857-1869. doi: 10.1038/s41564-022-01239-0
⁽⁴¹⁾ Sun Y, Guo X, Lu H, Chen L, Huang F, Liu Q, Feng Y. An Argonaute from Thermus parvatiensis exhibits endonuclease activity mediated by 5′ chemically modified DNA guides. Acta Biochim Biophys Sin (Shanghai). 2022 May 25; 54(5):686-695. doi: 10.3724/abbs.2022047

1. A method for screening for and/or identifying a nucleotide sequence of interest comprised in nucleic acids of a biological sample, comprising contacting at least a portion of the nucleic acids of the sample with a library of nucleic acid guides and a guide-dependent endonuclease, wherein the library of guides is obtained or derived from at least another portion of the same or a different sample, wherein the sequences of the guides align substantially without mismatches with the entirety of, or at least a portion of, nucleotide sequences that are expected to be present in the sample, wherein sample nucleic acid-endonuclease-guide complexes are formed and have endonuclease activity, and whereby expected nucleic acids in the sample are cleaved, and nucleic acid sequences in the sample which are not sufficiently complementary to any guide sequences are not cleaved.

2. A method as claimed in claim 1, wherein the nucleotide sequence of interest is (a) an unknown sequence; and/or (b) is of low abundance in the sample; and/or (c) is present in single copy; and/or (d) is a mutant allele.

3. A method as claimed in claim 1 or claim 2, further comprising a step of enrichment of sample nucleic acids; preferably wherein the enrichment comprises a capture-based enrichment.

4. A method as claimed in claim 3, wherein prior to the step of contacting, the sample or portion thereof is enriched for nucleic acid sequences present in at least a selected portion of the genome of an organism, or at least a selected portion of the transcriptome of an organism; optionally wherein the portion of sample used to generate the guides is enriched and/or the portion of the sample which is contacted is enriched.

5. A method as claimed in any of claims 1 to 4, further comprising an amplification reaction to increase the copy number of sample nucleic acids; preferably wherein the sample or portion thereof is subjected to amplification; optionally to increase the copy number of the nucleotide sequences of a selected portion of the genome or transcriptome of an organism.

6. A method as claimed in any preceding claim, wherein the source of the sample is selected from an organism, a cell culture or an environmental sample.

7. A method as claimed in any preceding claim, wherein the guides are prepared from a sample of nucleic acid from a first source, and wherein the sample or portion thereof contacted with (a) guides and a guided endonuclease, or (b) endonuclease-guide complexes, is from a sample of nucleic acid from a second source.

8. A method as claimed in claim 7, wherein the first source comprises a normal cell from an animal, and wherein the second source comprises a volume of blood from an animal.

9. A method as claimed in claim 8, wherein the first and second source is the same individual animal.

10. A method as claimed in claim 8 or claim 9, wherein the animal is a mammal; preferably a human.

11. A method as claimed in any preceding claim, wherein the guides are prepared from at least a portion of the or another sample by (i) fragmenting sample nucleic acids, (ii) taking a portion of the fragmented nucleic acids, (iii) hybridizing the portion of fragmented nucleic acids to a set of reference probes, wherein the reference probes are optionally shorter than the fragmented nucleic acids, (iv) digesting unhybridized single stranded nucleic acids to form double stranded nucleic acid fragment:probe hybrids, and (v) dissociating the double stranded hybrids so that the digested probes provide the single stranded guides.

12. A method as claimed in any preceding claim, wherein the guides consist of a multiplicity of sets of nucleic acid guides, and wherein separate portions of the sample are contacted with respective sets of nucleic acid guide-nuclease complexes.

13. A method as claimed in claim 12, wherein the sequences of one set of guides are different from the sequences of the other sets of guides.

14. A method as claimed in claim 12 or claim 13, wherein any resulting non-cleaved nucleic acid sequences are pooled.

15. A method as claimed in any of claims 1 to 6, wherein the guides are prepared from a separate portion of the sample taken from the same source as the portion of the sample which is taken and contacted with the guides and guide-dependent endonuclease.

16. A method as claimed in claim 15, wherein the separate portion of the sample is taken from the source at a first point in time, and the portion of sample contacted with the guides and guide-dependent endonuclease is taken at a second, later point in time.

17. A method as claimed in any of claims 11 to 16, wherein the source is a human sample; preferably a human blood sample and/or wherein the guides comprise between 1 and about 4800 equivalents of a double stranded genome known to be present in the source.

18. A method as claimed in any of claims 11 to 16, wherein the portion of the sample used to prepare the guides consists of not more than 50% of the weight of DNA in the sample.

19. A method as claimed in any of claims 11 to 16, wherein the nucleotide sequences of the guides consist of not more than 50% of the nucleotide sequences present in the sample.

20. A method as claimed in any preceding claim, wherein at least some of the sequences of the guides are known; optionally wherein the guides comprise a library of nucleic acid guides of known sequences.

21. A method as claimed in any preceding claim, wherein at least some of the sequences of the sample nucleic acids are known.

22. A method as claimed in any preceding claim, wherein guide-endonuclease complexes are formed first by contacting the nucleic acid guide fragments and endonuclease, and then the guide-endonuclease complexes are contacted with the sample.

23. A method as claimed in any preceding claim, wherein the guides are 5′ phosphorylated.

24. A method as claimed in any preceding claim, wherein the guides are targeted to a selected region or regions of a genome.

25. A method as claimed in any preceding claim, wherein the guides are of uniform length, optionally selected from a single length in the range of 8mers-50mers.

26. A method as claimed in any preceding claim, wherein the guides are DNA, and/or the sample comprises DNA.

27. A method as claimed in any preceding claim, wherein the nuclease is an Argonaute, preferably a prokaryotic Argonaute (pAgo); more preferably a pAgo from a thermophilic prokaryote; optionally a pAgo selected from Pyrococcus furiosus (PfAgo) or Thermus thermophilus (TtAgo).

28. A method as claimed in any preceding claim, further comprising the step of contacting nucleic acids from the sample with a library of guided nucleic acid-binding proteins that do not possess nuclease activity and which comprise a label or tag, wherein the protein-guide complexes bind to, but do not cleave respective nucleic acids which are other than a nucleotide sequence of interest, and wherein the protein-guide complexes and bound nucleic acids are separated from sample nucleic acids which are not bound to protein-guide complexes.

29. A method of preparing low abundance nucleotide sequences of interest present in a biological sample, comprising preparing nucleic acid according to a method of any of claims 1 to 28 and then subjecting the nucleic acid to a nucleic acid amplification reaction.

30. A method of determining presence of a nucleotide sequence of interest of unknown sequence present in a biological sample, comprising preparing nucleic acid according to a method of any of claims 1 to 28 and then subjecting the nucleic acid to polynucleotide sequencing.

31. A method as claimed in any preceding claim, wherein the nucleotide sequence of interest comprises a mutation; for example a mutation selected from one or more of a single nucleotide change, an insertion, a deletion or a duplication compared to a reference sequence; preferably wherein the mutation is a single nucleotide change.

32. A method as claimed in any preceding claim, wherein prior to contacting the sample nucleic acids with guides and guide-dependent endonucleases, either the guides or the nucleic acids from the biological sample are treated with a reagent that reacts with methylated base positions so that nucleotide sequences comprising methylated base positions are selectively preserved from guide sequence dependent endonuclease cleavage.

33. A method as claimed in any preceding claim wherein a computer is used in the processing and/or analysis of sequence data.
